SEQUENCE LISTING (103 sequences)

SEQ ID NO: 1 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWKDCFDQAVRGQDFGVSLTVDRPETVDTDDVLDKITRVLNESSFTERAEYYAGLLKAAGGRTAAADLLLGLPVLAND

SEQ ID NO: 2 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLCRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 3 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVHRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 4 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCKDQAVRGQDFGVSLTVDRPETVDTDDVLDKITRVLNESSFTERAEYYAGLLKAAGGRTAAADLLLGLPVLAND

SEQ ID NO: 5 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVKRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 6 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRADIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 7 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVCRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 8 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRARIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 9 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVPRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 10 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLNRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 11 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWGDCFDQAVRGQDFGVSLTVDRPETVDTDDVLDKITRVLNESSFTERAEYYAGLLKAAGGRTAAADLLLGLPVLAND

SEQ ID NO: 12 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSRVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 13 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCRDQAVRGQDFGVSLTVDRPETVDTDDVLDKITRVLNESSFTERAEYYAGLLKAAGGRTAAADLLLGLPVLAND

SEQ ID NO: 14 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCQDQAVRGQDFGVSLTVDRPETVDTDDVLDKITRVLNESSFTERAEYYAGLLKAAGGRTAAADLLLGLPVLAND

SEQ ID NO: 15 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCTDQAVRGQDFGVSLTVDRPETVDTDDVLDKITRVLNESSFTERAEYYAGLLKAAGGRTAAADLLLGLPVLAND

SEQ ID NO: 16 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWRDCFDQAVRGQDFGVSLTVDRPETVDTDDVLDKITRVLNESSFTERAEYYAGLLKAAGGRTAAADLLLGLPVLAND

SEQ ID NO: 17 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLFRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 18 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVSRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 19 - protein, 462 aa, synthetic construct
GAHRRPILFVSYAESGVLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 20 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVTRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 21 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVWRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 22 - protein, 462 aa, synthetic construct
GAHRRPILFVSYAESGLLNPLLVLAEELSRRGVEDLWFATDEKARDQIESASADSELQFASLGDTVSQMSADTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 23 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIEGMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 24 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVMRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 25 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLDRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 26 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAAIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 27 - protein, 462 aa, synthetic construct
GAHRRPILFVSYAESGLLNPLLVLAEELSRRGVEDLWFATDEKARDQIESASADSELQFASLGDTVSQMSAWTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 28 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVYRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 29 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWIDCFDQAVRGQDFGVSLTVDRPETVDTDDVLDKITRVLNESSFTERAEYYAGLLKAAGGRTAAADLLLGLPVLAND

SEQ ID NO: 30 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRATIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 31 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLQRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 32 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVGRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 33 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRACIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 34 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPILPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 35 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCHDQAVRGQDFGVSLTVDRPETVDTDDVLDKITRVLNESSFTERAEYYAGLLKAAGGRTAAADLLLGLPVLAND

SEQ ID NO: 36 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVDRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 37 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHAFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 38 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCCDQAVRGQDFGVSLTVDRPETVDTDDVLDKITRVLNESSFTERAEYYAGLLKAAGGRTAAADLLLGLPVLAND

SEQ ID NO: 39 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVQRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 40 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCWDQAVRGQDFGVSLTVDRPETVDTDDVLDKITRVLNESSFTERAEYYAGLLKAAGGRTAAADLLLGLPVLAND

SEQ ID NO: 41 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWEDCFDQAVRGQDFGVSLTVDRPETVDTDDVLDKITRVLNESSFTERAEYYAGLLKAAGGRTAAADLLLGLPVLAND

SEQ ID NO: 42 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVFRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 43 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCIDQAVRGQDFGVSLTVDRPETVDTDDVLDKITRVLNESSFTERAEYYAGLLKAAGGRTAAADLLLGLPVLAND

SEQ ID NO: 44 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVRRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 45 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCMDQAVRGQDFGVSLTVDRPETVDTDDVLDKITRVLNESSFTERAEYYAGLLKAAGGRTAAADLLLGLPVLAND

SEQ ID NO: 46 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCDDQAVRGQDFGVSLTVDRPETVDTDDVLDKITRVLNESSFTERAEYYAGLLKAAGGRTAAADLLLGLPVLAND

SEQ ID NO: 47 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVNRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 48 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWLDCFDQAVRGQDFGVSLTVDRPETVDTDDVLDKITRVLNESSFTERAEYYAGLLKAAGGRTAAADLLLGLPVLAND

SEQ ID NO: 49 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVVRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 50 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSAVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 51 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLIRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 52 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCLDQAVRGQDFGVSLTVDRPETVDTDDVLDKITRVLNESSFTERAEYYAGLLKAAGGRTAAADLLLGLPVLAND

SEQ ID NO: 53 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPHLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 54 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWMDCFDQAVRGQDFGVSLTVDRPETVDTDDVLDKITRVLNESSFTERAEYYAGLLKAAGGRTAAADLLLGLPVLAND

SEQ ID NO: 55 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAIIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 56 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGDLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 57 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIERMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 58 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVERHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 59 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIESMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 60 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVARHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 61 - protein, 462 aa, synthetic construct
GAHRRPILFVSYAESGLLNPLLVLAEELSRRGVEDLWFATDEKARDQIESASADSELQFASLGDTVSQMSATTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 62 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWSDCFDQAVRGQDFGVSLTVDRPETVDTDDVLDKITRVLNESSFTERAEYYAGLLKAAGGRTAAADLLLGLPVLAND

SEQ ID NO: 63 - protein, 462 aa, synthetic construct
GAHRRPILFVSYAESGLLNPLLVLAEELSRRGVEDLWFATDEKARDQIESASADSELQFASLGDTVSQMSAETWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 64 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVLRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 65 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAMIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 66 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRALIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 67 - protein, 462 aa, synthetic construct
GAHRRPILFVSYAESGLLNPLLVLAEELSRRGVEDLWFATDEKARDQIESASADSELQFASLGDTVSQMSAHTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 68 - protein, 462 aa, synthetic construct
GAHRRPILFVSYAESGLLNPLLVLAEELSRRGVEDLWFATDEKARDQIESASADSELQFASLGDTVSQMSALTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 69 - protein, 462 aa, synthetic construct
GAHRRPILFVSYAESGLLNPLLVLAEELSRRGVEDLWFATDEKARDQIESASADSELQFASLGDTVSQMSAYTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 70 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIEAMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 71 - protein, 462 aa, synthetic construct
GAHRRPILFVSYAESGLLNPLLVLAEELSRRGVEDLWFATDEKARDQIESASADSELQFASLGDTVSQMSANTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 72 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIEHMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 73 - protein, 462 aa, synthetic construct
GAHRRPILFVSYAESGLLNPLLVLAEELSRRGVEDLWFATDEKARDQIESASADSELQFASLGDTVSQMSAFTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 74 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPRLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 75 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHCFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 76 - protein, 462 aa, synthetic construct
GAHRRPILFVSYAESGLLNPLLVLAEELSRRGVEDLWFATDEKARDQIESASADSELQFASLGDTVSQMSACTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 77 - protein, 462 aa, synthetic construct
GAHRRPILFVSYAESGLLNPLLVLAEELSRRGVEDLWFATDEKARDQIESASADSELQFASLGDTVSQMSAQTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 78 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPLLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 79 - protein, 462 aa, synthetic construct
GAHRRPILFVSYAESGLLNPLLVLAEELSRRGVEDLWFATDEKARDQIESASADSELQFASLGDTVSQMSAITWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 80 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPALPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 81 - protein, 462 aa, synthetic construct
GAHRRPILFVSYAESGLLNPLLVLAEELSRRGVEDLWFATDEKARDQIESASADSELQFASLGDTVSQMSASTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 82 - protein, 462 aa, synthetic construct
GAHRRPILFVSYAESGLLNPLLVLAEELSRRGVEDLWFATDEKARDQIESASADSELQFASLGDTVSQMSAATWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 83 - protein, 462 aa, synthetic construct
GAHRRPILFVSYAESGLLNPLLVLAEELSRRGVEDLWFATDEKARDQIESASADSELQFASLGDTVSQMSAGTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 84 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMNPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 85 - protein, 462 aa, synthetic construct
GAHRRPILFVSYAESGLLNPLLVLAEELSRRGVEDLWFATDEKARDQIESASADSELQFASLGDTVSQMSAMTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 86 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHTFAPETRVEKYRALEKAVEEIQPALMVIECMCQFGYELAITKGIPFVLGVPMAPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 87 - protein, 462 aa, synthetic construct
GAHRRPILFVSYAESGLLNPLLVLAEELSRRGVEDLWFATDEKARDQIESASADSELQFASLGDTVSQMSARTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 88 - protein, 462 aa, synthetic construct
GAHRRPILFVSYAESGLLNPLLVLAEELSRRGVEDLWFATDEKARDQIESASADSELQFASLGDTVSQMSAKTWDDETYAEVTQRSRFKAHRAVIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 89 - protein, 462 aa, synthetic construct
GAHRRPILFVSYAESGLLNPLLVLAEELSRRGVEDLWFATDEKARDQIESASADSELQFASLGDTVSQMSAMTWDDETYAEVTQRSRFKAHRAVQRHTFAPETRVEKYRALEKAVEEIQPALMVIEHMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCRDQAVRGQDFGVSLTVDRPETVDTDDVLDKITRVLNESSFTERAEYYAGLLKAAGGRTAAADLLLGLPVLAND

SEQ ID NO: 90 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRASIRHTFAPETRVEKYRALEKAVEEIQPALMVIECMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 91 - protein, 462 aa, synthetic construct
GAHRRPILFVSYAESGLLNPLLVLAEELSRRGVEDLWFATDEKARDQIESASADSELQFASLGDTVSQMSAKTWDDETYAEVTQRSRFKAHRAVARHTFAPETRVEKYRALEKAVEEIQPALMVIECMCQFGYELAITKGIPFVLGVPALPSAVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCDDQAVRGQDFGVSLTVDRPETVDTDDVLDKITRVLNESSFTERAEYYAGLLKAAGGRTAAADLLLGLPVLAND

SEQ ID NO: 92 - protein, 462 aa, synthetic construct
GAHRRPILFVSYAESGLLNPLLVLAEELSRRGVEDLWFATDEKARDQIESASADSELQFASLGDTVSQMSAMTWDDETYAEVTQRSRFKAHRAVIRHTFAPETRVEKYRALEKAVEEIQPALMVIEHMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCMDQAVRGQDFGVSLTVDRPETVDTDDVLDKITRVLNESSFTERAEYYAGLLKAAGGRTAAADLLLGLPVLAND

SEQ ID NO: 93 - protein, 462 aa, synthetic construct
GAHRRPILFVSYAESGLLNPLLVLAEELSRRGVEDLWFATDEKARDQIESASADSELQFASLGDTVSQMSAMTWDDETYAEVTQRSRFKAHRAVARHTFAPETRVEKYRALEKAVEEIQPALMVIEHMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 94 - protein, 462 aa, synthetic construct
GAHRRPILFVSYAESGLLNPLLVLAEELSRRGVEDLWFATDEKARDQIESASADSELQFASLGDTVSQMSAMTWDDETYAEVTQRSRFKAHRAVIRHTFAPETRVEKYRALEKAVEEIQPALMVIEHMCQFGYELAITKGIPFVLGVPMLPSLVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 95 - protein, 462 aa, synthetic construct
GAHRRPILFVSYAESGLLNPLLVLAEELSRRGVEDLWFATDEKARDQIESASADSELQFASLGDTVSQMSAMTWDDETYAEVTQRSRFKAHRAVERHTFAPETRVEKYRALEKAVEEIQPALMVIEHMCQFGYELAITKGIPFVLGVPMLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCLDQAVRGQDFGVSLTVDRPETVDTDDVLDKITRVLNESSFTERAEYYAGLLKAAGGRTAAADLLLGLPVLAND

SEQ ID NO: 96 - protein, 462 aa, synthetic construct
GAHRRPILFVSYAESGLLNPLLVLAEELSRRGVEDLWFATDEKARDQIESASADSELQFASLGDTVSQMSAMTWDDETYAEVTQRSRFKAHRAVIRHTFAPETRVEKYRALEKAVEEIQPALMVIEHMCQFGYELAITKGIPFVLGVPLLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTLTRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 97 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHSFAPETRVEKYRALEKAVEEIQPALMVIECMCQFGYELAITKGIPFVLGVPNLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTITRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 98 - protein, 462 aa, synthetic construct
GAHRRPILFVSYAESGLLNPLLVLAEELSRRGVEDLWFATDEKARDQIESASADSELQFASLGDTVSQMSAVTWDDETYAEVTQRSRFKAHRAVIRHSFAPETRVEKYRALEKAVEEIQPALMVIECMCQFGYELAITKGIPFVLGVPTLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTITRLTREQVASLVEVARRLEGEGHQVLWKLPSEQQHLLPPAEELPANLRIESWVPSQLDVLAHPNVKVFFTHAGGNGYHEGLYFGKPLVVRPLWVDCFDQAVRGQDFGVSLTVDRPETVDTDDVLDKITRVLNESSFTERAEYYAGLLKAAGGRTAAADLLLGLPVLAND

SEQ ID NO: 99 - skipped

SEQ ID NO: 100 - protein, 462 aa, Streptomyces nodosus
GAHRRPILFVSYAESGLLNPLLVLAEELSRRGVEDLWFATDEKARDQIESASADSELQFASLGDTVSQMSAVTWDDETYAEVTQRSRFKAHRAVIRHSFAPETRVEKYRALEKAVEEIQPALMVIESMCQFGYELAITKGIPFVLGVPFLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTITRLTREQVASLVEVARRLEGEGHQVLWKLPSEQQHLLPPAEELPANLRIESWVPSQLDVLAHPNVKVFFTHAGGNGYHEGLYFGKPLVVRPLWVDCDDQAVRGQDFGVSLTVDRPETVDTDDVLDKITRVLNESSFTERAEYYAGLLKAAGGRTAAADLLLGLPVLAND

SEQ ID NO: 101 - protein, 479 aa, synthetic construct
MEKKIWSHPQFEKGGSGGAHRRPILFVSYAESGLLNPLLVLAEELSRRGVEDLWFATDEKARDQIESASADSELQFASLGDTVSQMSAVTWDDETYAEVTQRSRFKAHRAVIRHSFAPETRVEKYRALEKAVEEIQPALMVIESMCQFGYELAITKGIPFVLGVPFLPSNVLTSHVPFAKSYTPSGFPVPHSGLPGKMSLAQRVENELFRVRTLGMFMTKEIREIVEEDNRVRGELGISPEARQMMARIDHAEQVLCYSVAELDYPFPMHEKVRLVGTLVPPLPQAPDDEGLSDWLTEQKSVVFMGFGTITRLTREQVASLVEVARRLEGEGHQVLWKLPSEQQHLLPPAEELPANLRIESWVPSQLDVLAHPNVKVFFTHAGGNGYHEGLYFGKPLVVRPLWVDCDDQAVRGQDFGVSLTVDRPETVDTDDVLDKITRVLNESSFTERAEYYAGLLKAAGGRTAAADLLLGLPVLAND

SEQ ID NO: 102 - DNA, 22 bp, synthetic construct
  source 1..22: mol_type = other DNA
ctgagatacctacagcgtgagc

SEQ ID NO: 103 - DNA, 26 bp, synthetic construct
  source 1..26: mol_type = other DNA
cgtcactcatggtgatttctcacttg

(19) United States
(12) Patent Application Publication
(10) Pub. No.: US 2026/0009062 A1
(43) Pub. Date: Jan. 8, 2026

(54) TRANSFER OF C2'-EPIMERIZED SUGARS TO THE AMPHOTERICIN B AGLYCONE

(71) Applicants: The Board of Trustees of the University of Illinois, Urbana, IL (US); Northwestern University, Evanston, IL (US)

(72) Inventors: Martin D. Burke, Champaign, IL (US); Michael Christopher Jewett, Evanston, IL (US); Jonathan Webb Bogart, Evanston, IL (US); Saba Ghaffari, Champaign, IL (US)

(21) Appl. No.: 18/992,385

(22) PCT Filed: Jul. 10, 2023
(86) PCT No.: PCT/US2023/027248

§ 371 Date: Jan. 8, 2025

Related U.S. Application Data
(60) Provisional application No. 63/359,495, filed on Jul. 8, 2022.

(51) Int. Cl. C12P 19/18; C07H 17/08; C12N 9/10; C12P 19/62

(52) U.S. Cl. CPC C12P 19/18; C07H 17/08; C12N 9/1048; C12P 19/62

(57) ABSTRACT

Disclosed are polypeptides and methods of glycosylating the C19 hydroxyl group of AmdeB (i.e., amphotericin B lacking the sugar moiety) comprising contacting AmdeB with a saccharide in the presence of one of the polypeptides. The methods access compounds that are analogues of amphotericin B with a modified sugar moiety that have an improved therapeutic index, such as C2′epiAmB. Also disclosed are pharmaceutical compositions comprising the compounds and a pharmaceutically acceptable carrier.

### RELATED APPLICATIONS

The application claims the benefit of priority to U.S. Provisional Patent Application Ser. No. 63/359,495, filed Jul. 8, 2022.

### FIELD

The present disclosure provides polypeptides, and methods of using the polypeptides to prepare analogues of amphotericin B. More particularly, the present disclosure relates to polypeptides and methods of glycosylating the C19 hydroxyl group of AmdeB (i.e., amphotericin B lacking the mycosamine sugar moiety), comprising combining AmdeB with a saccharide in the presence of one of the polypeptides. The methods access compounds that are analogues of amphotericin B with a modified sugar moiety, which analogues have an improved therapeutic index, such as C2′epiAmB.

### BACKGROUND

Amphotericin B (AmB) has served as the gold standard for the treatment of life-threatening systemic fungal infections for more than half a century, and in stark contrast to many antibiotics, resistance to AmB remains exceptionally rare. Despite high potency and broad-spectrum antifungal activity, AmB is highly toxic to humans. Consequently, dose-limiting side effects can preclude the effective treatment of fungal infections with AmB.

Key structure-activity-relationships revealed that amphotericin's toxicity can be attributed to a unique small molecule-small molecule interaction, coordinated in large part by the unusual mycosamine sugar on the natural product (See K. C. Gray et al., PNAS 2012, 109, 2234). Indeed, removal of mycosamine from AmB (AmdeB) completely abolishes cell-killing activity in both yeast and human cell assays (See D. S. Palacios et al., J Am Chem Soc 2007, 129, 13804).

Modifying the structure of the sugar moiety of AmB has provided analogues with reduced human toxicity but retained antifungal activity. One analogue showing particular promise is C2′epi-amphotericin B (C2′epiAmB, shown below).

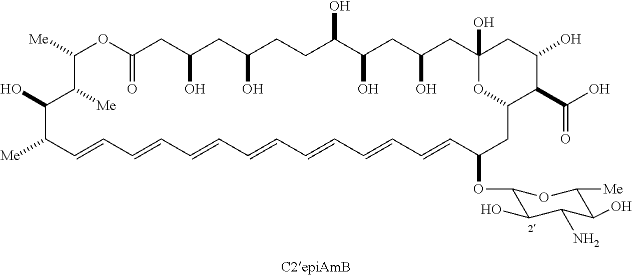 [SMILES: CC1OC(O[C@H]2/C=C/C=C/C=C/C=C/C=C/C=C/C=C/[C@H](C)[C@@H](O)[C@@H](C)[C@H](C)OC(=O)C[C@H](O)C[C@H](O)CC[C@@H](O)[C@H](O)C[C@H](O)C[C@]3(O)C[C@H](O)[C@@H](C(=O)O)[C@H](C2)O3)C(O)C(N)C1O]

C2′epiAmB retains potent antifungal activity and is orders of magnitude less toxic than AmB (See, e.g., WO 2016/061437A1). Despite the improved therapeutic index of C2′epiAmB and other AmB analogues, challenges associated with producing these complex structures on industrial scale by chemical synthesis has limited their viability as practical AmB replacements. Accordingly, there is a need for improved methods to produce C2′epiAmB and other AmB analogues.

Further, synthetic challenges have limited the opportunity to modify the sugar moiety of AmB and thus discover additional AmB analogues having improved therapeutic properties. Therefore, there is a need for methods that provide access to new analogues of amphotericin B with a modified sugar moiety.

### SUMMARY OF THE INVENTION

In certain aspects, provided is a polypeptide, or a salt thereof, comprising an amino acid sequence having at least 95% sequence identity to the amino acid sequence of any one of SEQ ID NOs.: 1-98.

In some aspects, provided is a method of glycosylating the C19 hydroxyl group of AmdeB, comprising the step of combining under conditions sufficient to glycosylate the C19 hydroxyl group of AmdeB:
(i) AmdeB, or a salt thereof;

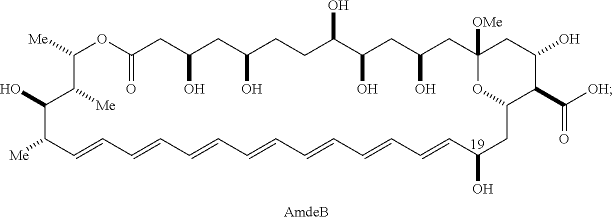

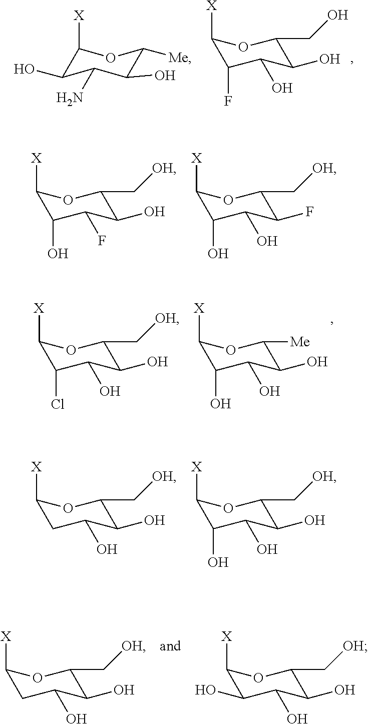

(ii) a saccharide selected from the group consisting of:
   wherein X is an oxygen-linked nucleoside diphosphate; and
(iii) a polypeptide, or a salt thereof, comprising an amino acid sequence having at least 95% sequence identity to the amino acid sequence of any one of SEQ ID NOs.: 1-98.

In certain aspects, provided is a compound, or a pharmaceutically acceptable salt thereof, selected from the group consisting of:

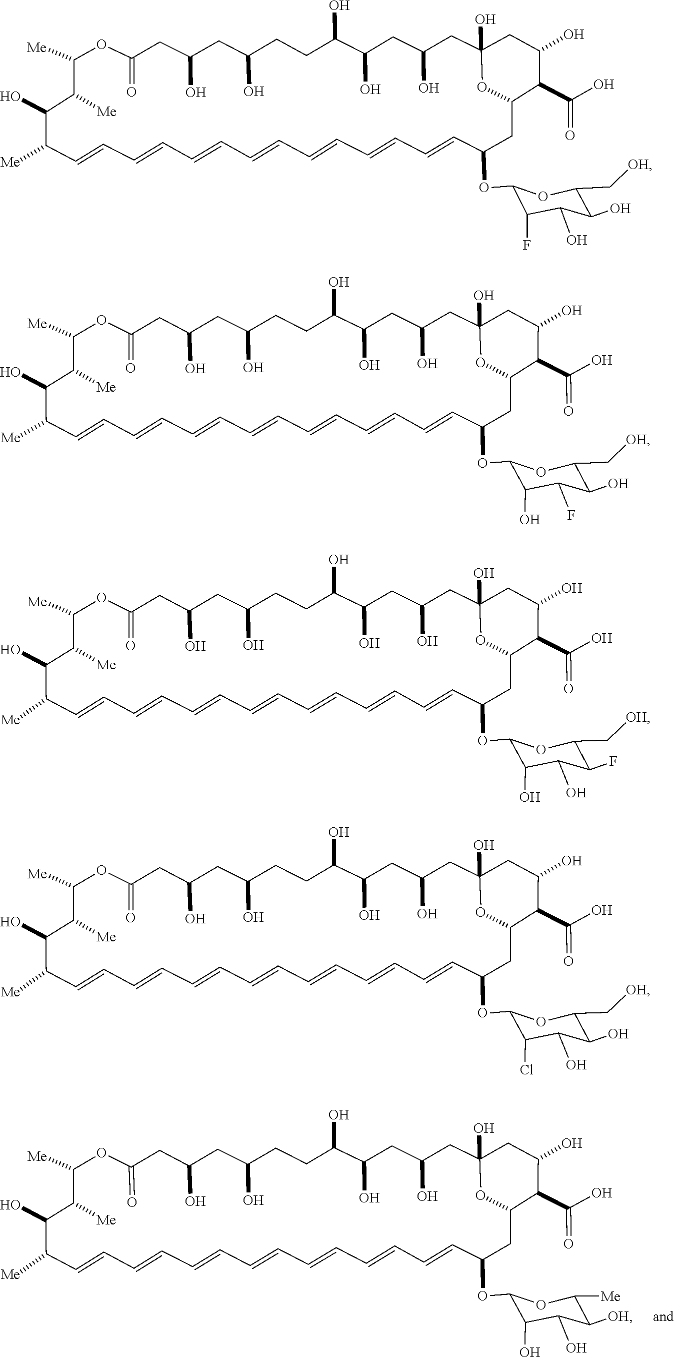

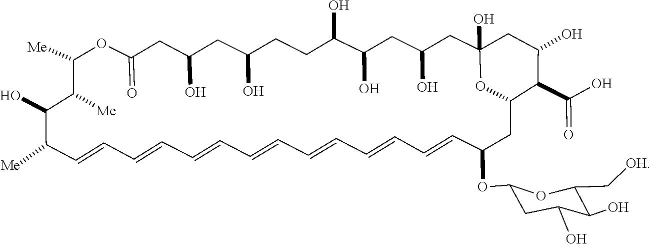 [SMILES: C[C@@H]1[C@H](O)[C@@H](C)/C=C/C=C/C=C/C=C/C=C/C=C/C=C/[C@H](OC2CC(O)C(O)C(CO)O2)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)O]

In some aspects, provided is a pharmaceutical composition, comprising any one of the compounds; and a pharmaceutically acceptable carrier.

### BRIEF DESCRIPTION OF THE DRAWINGS

FIG. 1 shows a substrate sequence employed to evolve mutants of AmB's natural glycosyltransferase (AmphDI) to have activity for transferring the unnatural sugar C2′epimycosamine to the amphotronolide acceptor (AmdeB).

FIG. 2 shows various AmB analogues formed by the transfer of sugars to AmdeB with top AmphiDI mutants from the GDP-mannose campaign.

FIG. 3 shows a schematic of an idealized cell-free, machine learning-guided protein engineering workflow.

FIG. 4 shows the conversion percentage to C2′epiAmB obtained with a selection of active mutants. Data from Round 1 (R1) correspond to the entries beginning at the origin and progressing along the x-axis. The seven entries at the far end of the x-axis correspond to data from Round 2 (R2).

### DETAILED DESCRIPTION OF THE INVENTION

Microbes are extraordinarily adept at producing AmB, as metric tons are fermented annually. Enzymes found within amphotericin's natural biosynthetic pathway can serve as exceptionally specific and renewable biocatalysts but are currently incapable of accessing known non-toxic variants, such as C2′epiAmB.

Given the staggering success of repurposing and engineering natural biosynthetic machinery to produce complex molecules, the inventors sought to similarly develop a biosynthetic strategy to manufacture C2′epiAmB. A critical piece to realizing a fully biocatalytic strategy is to identify an enzyme capable of transferring the unnatural sugar, C2′epimycosamine, to the amphotronolide acceptor (AmdeB).

AmB's natural glycosyltransferase (AmphDI) displays a relatively strict substrate scope of only 3 sugars, and displays no detectable activity for transferring C2′epimycosamine despite only a single stereochemical switch from the natural sugar. Although AmphDI provided no starting point from which a mutant could be engineered with the desired activity, the inventors used a substrate walking protein engineering approach in combination with a machine learning-guided protein engineering workflow to surprisingly discover that a combination of several mutations to AmphDI provided enzymes capable of transferring C2′epimycosamine to AmdeB.

Additionally, some of the AmphDI mutants involving multiple substitutions displayed a dramatically expanded the substrate scope to include sugars with a range of heteroatoms and stereochemistries not tolerated by the natural enzyme. Therefore, these AmphDI mutants were able to access AmB analogues with a modified sugar moiety that may have an improved therapeutic index.

In some aspects, a polypeptide, or a salt thereof, comprising an amino acid sequence having at least 95% sequence identity to the amino acid sequence of any one of SEQ ID NOs.: 1-98 is provided.

In some embodiments, the polypeptide comprises an amino acid sequence having at least 96%, at least 97%, at least 98%, at least 99%, or 100% sequence identity to the amino acid sequence of any one of SEQ ID NOs.: 1-98.

In certain embodiments, the polypeptide comprises the amino acid sequence of any one of SEQ ID NOs.: 1-98.

In some embodiments, the polypeptide comprises an amino acid sequence having at least 95% sequence identity to the amino acid sequence of any one of SEQ ID NOs.: 73-89, 91, and 92.

In certain embodiments, the polypeptide comprises an amino acid sequence having at least 95% sequence identity to the amino acid sequence of any one of SEQ ID NOs.: 86-89, 91, and 92.

In some embodiments, the polypeptide comprises an amino acid sequence having at least 95% sequence identity to the amino acid sequence of any one of SEQ ID NOs.: 87-89 and 91.

In some embodiments, the polypeptide comprises an amino acid sequence having at least 95%, at least 96%, at least 97%, at least 98%, at least 99%, or 100% sequence identity to the amino acid sequence of SEQ ID NO: 88.

In certain embodiments, the polypeptide comprises an amino acid sequence having at least 95%, at least 96%, at least 97%, at least 98%, at least 99%, or 100% sequence identity to the amino acid sequence of SEQ ID NO: 89.

In certain aspects, provided is a method of glycosylating the C19 hydroxyl group of AmdeB, comprising the step of combining under conditions sufficient to glycosylate the C19 hydroxyl group of AmdeB:
(i) AmdeB, or a salt thereof;

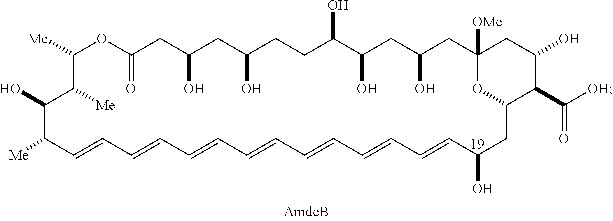

(ii) a saccharide selected from the group consisting of:

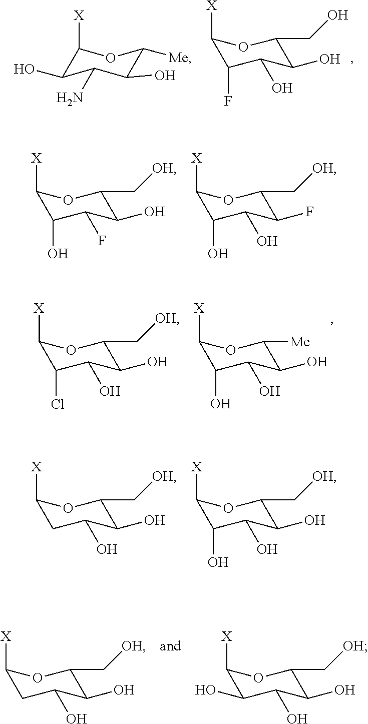

wherein X is an oxygen-linked nucleoside diphosphate; and
(iii) a polypeptide, or a salt thereof, comprising an amino acid sequence having at least 95% sequence identity to the amino acid sequence of any one of SEQ ID NOs.: 1-98.

In some embodiments, X is oxygen-linked guanosine diphosphate, adenosine diphosphate, cytosine diphosphate, uridine diphosphate, or thymidine diphosphate. In some embodiments, X is oxygen-linked guanosine diphosphate or uridine diphosphate. In certain embodiments, X is oxygen-linked guanosine diphosphate.

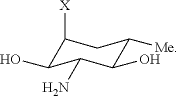

In certain embodiments, the saccharide is

In certain embodiments, the molar ratio of the saccharide to the polypeptide is from about 10,000:1 to about 100:1. In some embodiments, the molar ratio of AmdeB to the polypeptide is from about 10:1 to about 20:1.

In certain aspects, provided is a compound, or a pharmaceutically acceptable salt thereof, selected from the group consisting of:

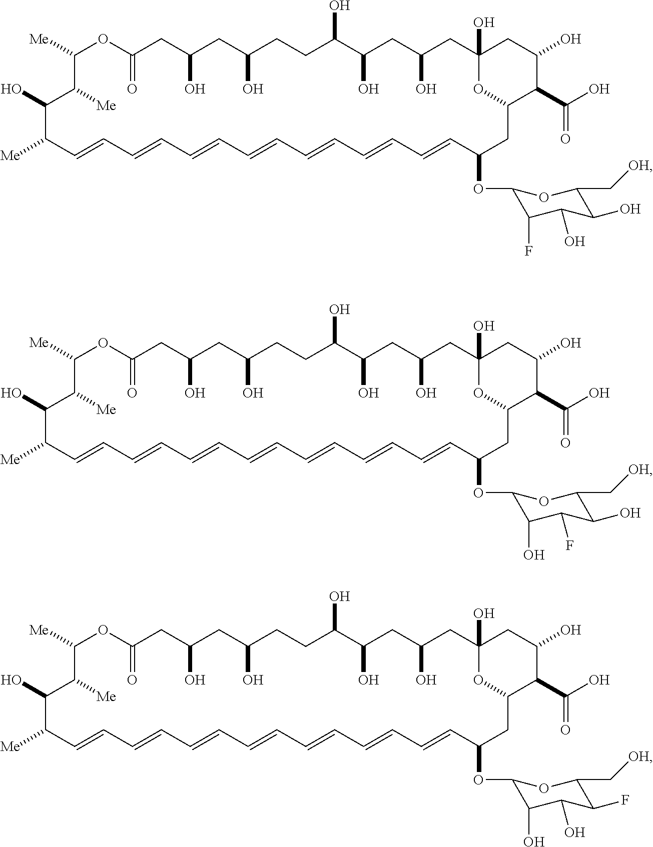

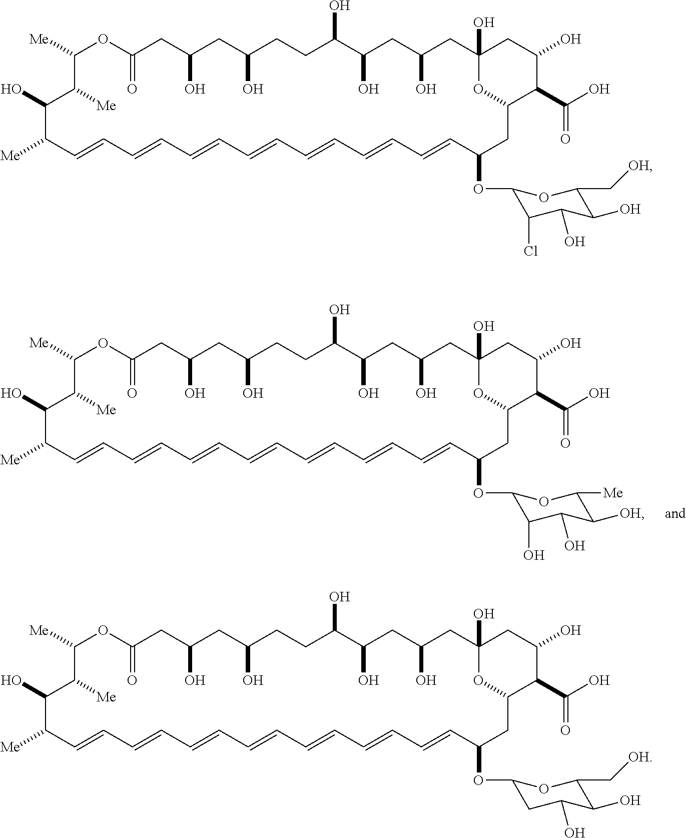

In some embodiments, the compound is selected from the group consisting of:

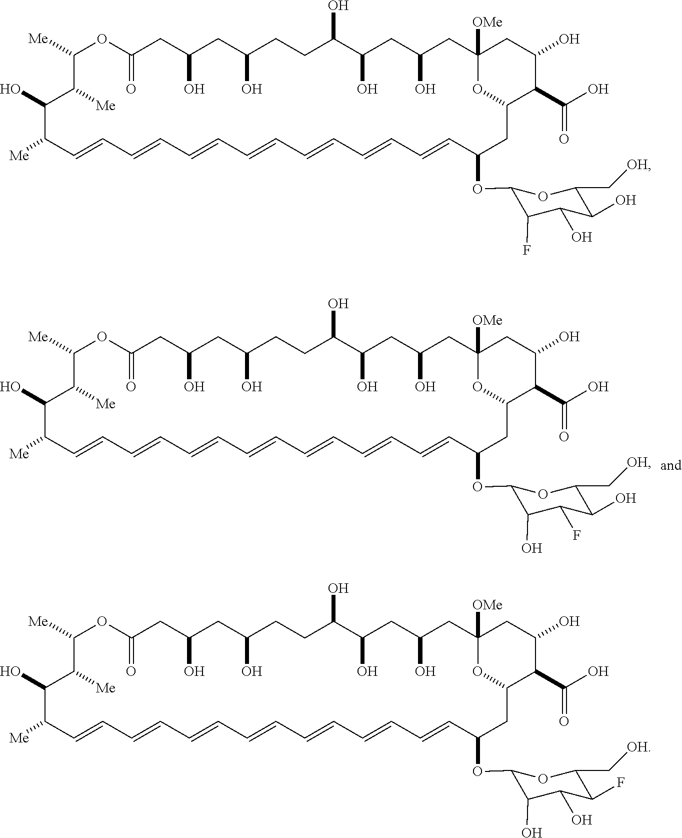

In certain embodiments, the compound is selected from the group consisting of:

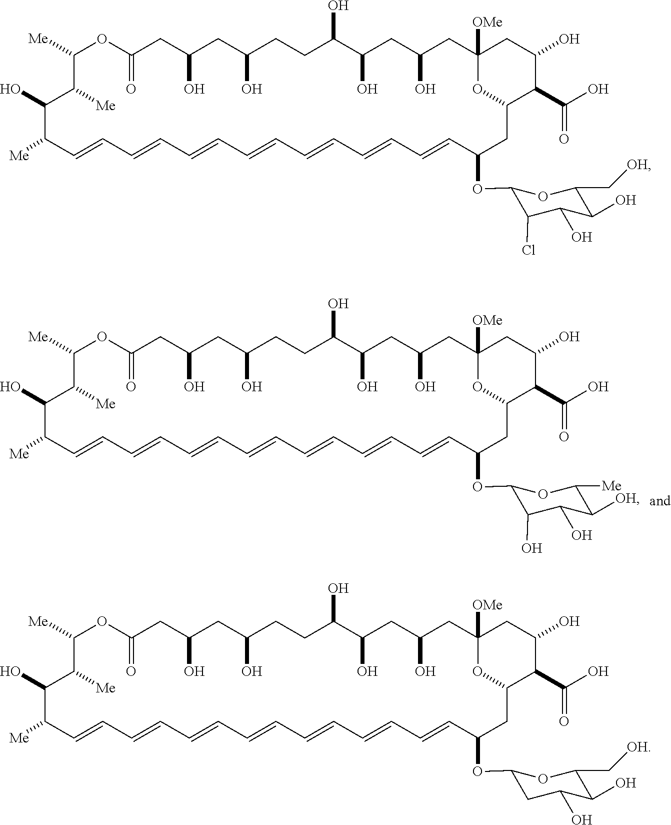

In some embodiments, the compound is selected from the group consisting of:

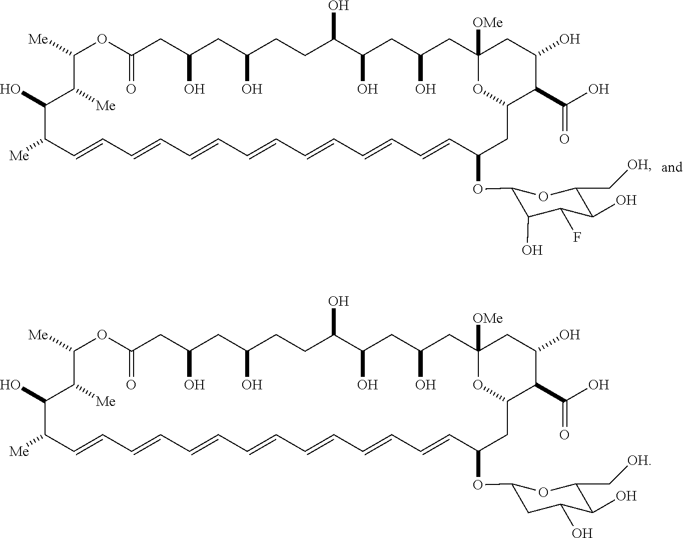

In some aspects, provided is a pharmaceutical composition comprising the compound, and a pharmaceutically acceptable carrier.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The term “AmphDI” means AmB's natural glycosyltransferase comprising the amino acid sequence of SEQ ID NO.: 100 corresponding to Amino Acids 22-483 of the full domain (see https://www.uniprot.org/uniprotkb/Q93NW9). SEQ ID NO.: 100 is alternatively referred to herein as the AmphDI wild type sequence. Table 1 discloses this sequence.

TABLE 1
AmphDI Amino Acid Sequence

| | | SEQ ID |
|---|---|---|
| Domain | Amino Acid Sequence | NO: |
| Amino Acids 22-483 | GAHRRPILFVSYAESGLLNPLLVLAEELSRRGV | 100 |
| of the full domain | EDLWFATDEKARDQIESASADSELQFASLGDT | |
| | VSQMSAVTWDDETYAEVTQRSRFKAHRAVIR | |
| | HSFAPETRVEKYRALEKAVEEIQPALMVIESMC | |
| | QFGYELAITKGIPFVLGVPFLPSNVLTSHVPFAK | |
| | SYTPSGFPVPHSGLPGKMSLAQRVENELFRVRT | |
| | LGMFMTKEIREIVEEDNRVRGELGISPEARQM | |
| | MARIDHAEQVLCYSVAELDYPFPMHEKVRLVG | |
| | TLVPPLPQAPDDEGLSDWLTEQKSVVFMGFGTI | |
| | TRLTREQVASLVEVARRLEGEGHQVLWKLPSE | |
| | QQHLLPPAEELPANLRIESWVPSQLDVLAHPNV | |
| | KVFFTHAGGNGYHEGLYFGKPLVVRPLWVDC | |
| | DDQAVRGQDFGVSLTVDRPETVDTDDVLDKIT | |
| | RVLNESSFTERAEYYAGLLKAAGGRTAAADLL | |
| | LGLPVLAND | |

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

The term “pharmaceutically acceptable carrier” means one or more compatible solid or liquid filler, diluent, or encapsulating substances which are suitable for administration to a human or other vertebrate animal. The term “carrier” denotes an organic or inorganic ingredient, natural or synthetic, with which the active ingredient is combined to facilitate the administration. The components of the compositions also are capable of being commingled in a manner such that there is no interaction which would substantially impair the desired pharmaceutical efficacy.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)-or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In certain embodiments, the weights are based upon total weight of compound.

As used herein and unless otherwise indicated, the term “enantiomerically pure S-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)-or(S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

The term “about” refers to variations in numerical values typically encountered by one of skill in the art of respirable formulations, including variations of plus or minus 0.1%, 0.5%, 1%, 2%, 3%, 4%, 5%, 6%, 7%, 8%, 9%, or 10% of a numerical value described herein.

The term “plasmid,” refers to a circular double stranded DNA loop into which another DNA segments may be ligated.

### EXAMPLES

Having now described the present invention in detail, the same will be more clearly understood by reference to the following examples, which are included herewith for purposes of illustration only and are not intended to be limiting of the invention.

### Materials and Methods

### Cell-Free Library Generation and Protein Synthesis

Primers for mutagenesis using a revised cell-free protein engineering method were designed using Benchling with melting temperature calculated by the default SantaLucia 1998 algorithm. Melting temperatures of alternative primer design tools sometimes deviate greatly from those calculated in Benchling, so users should consider this when designing primers. The general heuristics followed for primer design were a reverse primer of 58° C., a forward primer of 62° C., and a homologous overlap of approximately 45° C. All primers were ordered from Integrated DNA Technologies (IDT); forward primers were synthesized in 384-well plates normalized to 2-μM for ease of setting up reactions.

The codons in Table 2 were used in the forward primers in our cell-free DNA assembly workflow to mutate a desired residue into the corresponding amino acid. While the addition of excess tRNA in CFPS reactions mitigates the negative effects of unoptimized codons, the most prevalent codon found in E. coli was used for the compatibility of in vivo expression and to prevent the need for re-optimizing the entire sequence.

TABLE 2
Codons Used in the Forward Primers
in Cell-Free DNA assembly workflow

| Amino Acid | Codon |
|---|---|
| A | GCG |
| R | CGT |
| N | AAC |
| D | GAT |
| C | TGC |
| Q | CAG |
| E | GAA |
| G | GGC |
| H | CAT |
| I | ATT |
| L | CTG |
| K | AAA |
| M | ATG |
| F | TTT |
| P | CCG |
| S | AGC |
| T | ACC |
| W | TGG |
| Y | TAT |
| V | GTG |

All cloning steps were set up using an Integra VIAFLO liquid handling robot in 384-well PCR plates (Bio-Rad). The cell-free library generation was performed as follows: (1) the first PCR was performed in a 10-μL reaction with 1-ng of plasmid template added, (2) 1-μL of DpnI was added and incubated at 37° C. for two hours, (3) the PCR was diluted 1:4 by the addition of 29-μL of nuclease-free (NF) water, (4) 1-μL of diluted DNA was added to a 3-μL Gibson assembly reaction and incubated for 50° C. for one hour, (5) the assembly reaction was diluted 1:10 by the addition of 36-μL of NF water, (6) 1-μL of the diluted assembly reaction was added to a 9-μL PCR reaction. All PCR reactions used Q5 Hot Start DNA Polymerase (NEB).

The thermocycler parameters in Table 3 and Table 4 were consistent throughout this study, with extension time being the only variable changing to compensate for different amplicon lengths. The first step uses touchdown PCR, in which the initial annealing temperature decreases by 1° C. each cycle until a final set temperature is reached.

TABLE 3
PCR 1 parameters

| Step | Temp (° C.) | Time (min:sec) |
|---|---|---|
| Initial | 98 | 3:00 |
| Denaturation | | |
| 6x | 98 | 0:30 |
| | 70 (−1° C./cycle) | 0:30 |
| | 72 | 20 s/kbp |
| 20x | 98 | 0:30 |
| | 64 | 0:30 |
| | 72 | 20 s/kbp |
| Final Extension | 72 | 10:00 |
| Hold | 12 | ∞ |

TABLE 4
PCR 2 parameters

| Step | Temp (° C.) | Time (min:sec) |
|---|---|---|
| Initial | 98 | 3:00 |
| Denaturation | | |
| 30x | 98 | 0:30 |
| | 68 | 0:30 |
| | 72 | 20 s/kbp |
| Final Extension | 72 | 10:00 |
| Hold | 12 | ∞ |

The primers in Table 5 are universally used to amplify LETs off pJL1 containing any gene of interest. They add approximately 300 basepairs both upstream and downstream of the coding region to help protect against exonucleases present in the E. coli lysate.

TABLE 5
Primers Used to Amplify LETs off PJL1

| Direction | Sequence |
|---|---|
| LET_fwd | CTGAGATACCTACAGCGTGAGC |
| LET_rvs | CGTCACTCATGGTGATTTCTCACTTG |

To accumulate mutations for ISM, 3-μL of the “winner” from the diluted Gibson assembly plate was transformed into 20-μL of chemically competent E. coli (NEB 5-alpha cells). Cells were plated onto LB plates containing 50 μg/mL kanamycin (LB-Kan). A single colony was used to inoculate a 50 mL overnight culture of LB-Kan, grown at 37° C. with 250 RPM shaking. The plasmid was purified using ZymoPURE II Midiprep kits and sequence confirmed.

Crude cell extracts were prepared using E. coli BL21 Star (DE3) cells (Invitrogen). CFPS reactions were performed based on the Cytomim system and carried out in 384-well PCR plates (Bio-Rad) as 10-μL reactions with 1-μL of LET serving as the DNA template. AmphDI from Streptomyces nodosus (UniProt: Q93NW9) was codon-optimized for E. coli and cloned into the pJL1 plasmid with an N-terminal CSL-tag (CAT-Strep-Linker fusion containing Strep-tag II).

### Forward, Glycosylation Activity Assay

All high-throughput assays (hot spot screen, iterative site saturation mutagenesis, substrate scope, ML predictions validation, and ML prediction exploration) were assembled in 384-well plates (Bio-Rad) using an Integra VIAFLO liquid handling robot. A 2× reaction mix containing the substrates (MgCl₂, AmdeB, and NDP-sugar) with excess volume filled with 50 mM Tris HCl pH 8.0 was dispensed as 3-μL aliquots in a 384-well plate. The glycosylation assay was initiated by adding 3-μL of crude CFPS reaction containing an expressed AmphDI variant, with final concentrations of 10 mM MgCl₂, 75 uM AmdeB, 1-50 mM NDP-sugar (depending on the stage of the campaign and sugar type), 1% v/v DMSO (from AmdeB stock), and ˜5 μM of enzyme (determined by 14C-leucine incorporation using previously described protocols). Stock solutions of the AmdeB were prepared in DMSO and this was taken into account to reach 1% v/v DMSO. For reactions that were performed in triplicates, 3-μL from the same 10-μL CFPS reaction was used for three separate assays. The reaction was incubated at 37° C. for 16 hours and then quenched with 25-μL of methanol. Plates were stored at −20° C. until prepared for analysis.

### Reverse, Glycosylation Activity Assay

All reverse reactions were assembled in 384-well plates (Bio-Rad) using an Integra VIAFLO liquid handling robot. A 2× reaction mix containing the substrates (MgCl₂, AmB or C2′epiAmB, and GDP) with excess volume filled with 50 mM Tris HCl pH 8.0 was dispensed as 3-μL aliquots in a 384-well plate. The glycosylation assay was initiated by adding 3-μL of crude CFPS reaction containing an expressed AmphDI variant, with final concentrations of 10 mM MgCl₂, 75 μM AmB or C2′epiAmB, 35 mM GDP, and ˜5 μM of enzyme (determined by 14C-leucine incorporation using previously described protocols). Stock solutions of the AmB and C2′epiAmB were prepared in DMSO and this was taken into account to reach 1% v/v DMSO. For reactions that were performed in triplicates, 3-μL from the same 10-μL CFPS reaction was used for three separate assays. The reaction was incubated at 37° C. for 16 hours and then quenched with 25-μL of methanol. Plates were stored at −20° C. until prepared for analysis.

### Analytics

All products were analyzed using an Agilent G6125B Single Quadrupole LC/MSD system equipped with an electrospray ionization source set to positive ionization mode. The quenched samples were centrifuged for 10 min at 4,500×g to remove precipitated proteins. A separate 384-well plate for sample injection into the HPLC-MS was prepared by dispensing 25 μL of the quenched samples into it using the Integra VIAFLO. Trace amounts of compounds were detected using MS, while many compounds were present in high enough concentration to quantify by diode array detector (DAD) at 254 and 406 nm. Compounds were separated on a Luna C18 Column (Phenomenex 00D-4251-B0) using mobile phases (A) H₂O with 0.1% formic acid and (B) Acetonitrile. The general method for chromatographic separation was carried out using the following gradients at a constant flow rate of 0.5 mL/min: 0 min 30% B; 0.5 min 50% B; 2.0 min 62% B; 2.25 min 95% B; 2.45 min 95% B; 2.5 min 10% B; 2.65 min 10% B; 2.7 min 30% B; 3.0 min 30% B. For the MS, capillary voltage was set at 3 kV, and nitrogen gas was used for nebulizing (35 psig) and drying (12 1/min, 350° C.). The MS was calibrated using Tuning Mix (Agilent G2421-60001) before measurements were taken. MS data were acquired with a scan range of 50-600 m/z with various SIM m/z's according to which compound we were screening for. LC-MS data were collected and analyzed using Agilent OpenLab CDS ChemStation software. The product yield was calculated by dividing the DAD peak area for the amide product by the sums of the peak areas of both the amide and the acid substrate.

### Expression and Purification of Recombinant Proteins

All proteins in this study were purified according to their literature precedent or by the method described below. All AmphDIs (including all mutants) plasmid was transformed into chemically competent E. coli BL21 Star (DE3) cells (Invitrogen) following the manufacturer's instructions. Cells were plated onto LB-Kan and incubated overnight at 37° C. A single colony was used to inoculate a 5 mL overnight culture of LB-Kan, grown at 37° C. with 250 RPM shaking. 1 L of Overnight Express TB Medium (Millipore) was prepared following the manufacturer's instructions and supplemented with 100 μg/mL kanamycin. The TB medium was inoculated the following day using the 5 mL overnight culture and grown at 37° C. with 250 RPM shaking until saturation (˜ 12-16 hours). Cells were harvested by centrifugation (Beckman Coulter Avanti J-26) at 8,000×g for 10 min at 4° C. Cell pellets were either flash frozen with liquid nitrogen and stored at −20° C. until future use or resuspended in 25 mL Wash Buffer (100 mM Tris-HCl pH 8.0, 150 mM NaCl, 1 mM EDTA, 10% v/v glycerol). Resuspended cells were lysed by sonication (QSonica Q700 Sonicator) using six 10 seconds ON and 10 seconds OFF cycles at 50% amplitude, and the insoluble fraction was removed by centrifugation at 12,000×g for 20 minutes at 4° C. Clarified lysates were incubated with 2 mL of pre-equilibrated Strep-Tactin XT Superflow resin (IBA Lifesciences) with shaking for 30 min at 4° C. Resin was loaded onto a gravity-flow column and washed three times with 20 mL Wash Buffer. AmphDI protein was eluted with 10 mL of Elution Buffer (100 mM Tris-HCl pH 8.0, 150 mM NaCl, 1 mM EDTA, 50 mM biotin, 10% v/v glycerol) and concentrated with a 15 mL Amicon Ultra Centrifugal filter (Millipore Sigma; 30 kDa cutoff). Purified AmphDI was buffer exchanged into Storage Buffer (50 mM HEPES pH 7.5, 300 mM NaCl, 10 mM MgCl₂, 10% v/v glycerol) using a pre-equilibrated PD-10 desalting column (Cytiva). AmphDI was stored at 4° C. for immediate use (<48 hours) or −20° C. for longer term storage. Protein concentration was quantified by measuring A280 on a NanoDrop 2000c (Thermo Scientific), with AmphDI extinction coefficient and molecular weight calculated by Expasy ProtParam.

### Example 1: Polyene Glycosyltransferases have No Native Activity for C2′Epimycosamine

An initial objective was to determine if any natural glycosyltransferase (GT) could accept AmdeB and GDP-C2′epimycosamine as substrates. 144 unique polyene GTs were identified, including the natural amphotericin B GT (AmphDI), that may exhibit this desired activity. Many polyene glycosyltransferases (GTs) decorate natural products by using nucleotide diphosphate sugars (NDP-sugars) as activated donors and expel NDP as a result of glycosidic bond formation. Reactions can be pushed in the reverse direction, effectively deglycosylating the natural product, using the native GT and excess NDP. Given the synthetically complexity of the GDP-C2′epimycosamine, it was reasoned that challenging each enzyme to perform the reverse reaction on a synthetic standard of C2′epiAmB may be a simpler way of assessing if the enzymes had the desired activity. NDP-sugar formation is thermodynamically disfavored (Keq<1) and yields are often poor (<10%), however reactions can be pushed by excess NDP and yields should be high enough to identify even weak activity.

All enzymes were purchased as fully formed plasmids and expressed in analytical amounts using standard cell-free protein synthesis. All enzymes were also tested for activity with both authentic AmB and C2′epiAmB. The initial results with (cell-free expressed) AmphDI are consistent with the literature as conversion to the AmdeB plateaus at 10% after 2 hours. Yields can be pushed further to 20% by increasing GDP, but screens of enzyme loading, temperature, buffer, pH, cofactor and substrate failed to increase yields further. In total, seven homologs in addition to the natural AmphDI were active for AmB, with AmphDI displaying the highest overall activity. All enzymes failed to catalyze the reverse reaction with C2′epiAmB. From this data a protein engineering campaign was initiated with AmphDI as the parent sequence.

### Example 2: Substrate Walking Approach and GDP-Mannose Campaign

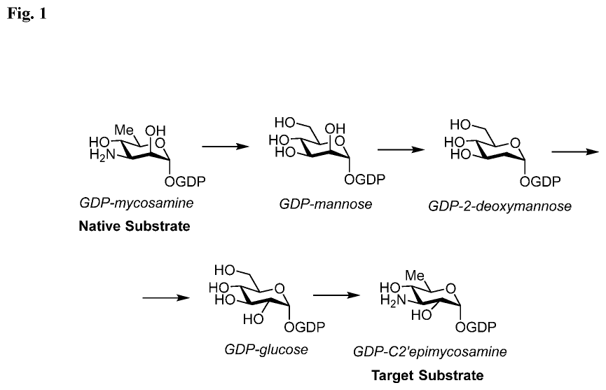

Despite the strict substrate scope of AmphDI, it was reasoned that a substrate walking approach could eventually lead to the desired activity. To this end a hypothetical evolutionary pathway going from natural mycosamine>mannose>2-deoxymannose>glucose>C2′epimycosamine was devised (see FIG. 1 for an illustration). Mannose was used for the first intermediate sugar because it was one of only two unnatural sugars AmphDI was known to tolerate. Furthermore, GDP-mannose was commercially available making it the only practical starting point given the large quantities that would be needed for initial screens. From there, a jump to 2-deoxymannose and glucose may be possible and would focus a second screen towards residues that influence specificity relative to the important 2′ position. Next, glucose could serve as a potential third intermediate sugar and the first to display the intended stereochemistry at the 2′ position and an engineering campaign may lead to weak activity for the desired C2′epimycosamine.

For the initial screen a homology model of AmphDI was constructed and 96 residue positions within ˜5 A of the putative active site were selected for substitutions, encompassing nearly 20% of the enzyme.

The residue positions selected for substitutions were 31, 32, 33, 34, 36, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 143, 144, 145, 146, 147, 164, 165, 166, 167, 168, 169, 170, 171, 172, 173, 174, 189, 190, 191, 192, 193, 241, 242, 243, 244, 245, 246, 285, 306, 307, 308, 309, 310, 311, 338, 340, 361, 362, 363, 364, 365, 366, 380, 381, 382, 383, 384, 385, 386, 389, 400, 401, 402, 403, 404, 405, 406, 407, and 408.

These residues are numbered relative to the wild-type AmPhDI with a 17-amino acid residue tag. The tagged wild-type polypeptide has the following sequence:

(SEQ ID NO.: 101)
MEKKIWSHPQFEKGGSGGAHRRPILFVSYAESGLLNPLLVLAEELSRR
GVEDLWFATDEKARDQIESASADSELQFASLGDTVSQMSAVTWDDETY
AEVTQRSRFKAHRAVIRHSFAPETRVEKYRALEKAVEEIQPALMVIES
MCQFGYELAITKGIPFVLGVPFLPSNVLTSHVPFAKSYTPSGFPVPHS
GLPGKMSLAQRVENELFRVRTLGMFMTKEIREIVEEDNRVRGELGISP
EARQMMARIDHAEQVLCYSVAELDYPFPMHEKVRLVGTLVPPLPQAPD
DEGLSDWLTEQKSVVFMGFGTITRLTREQVASLVEVARRLEGEGHQVL
WKLPSEQQHLLPPAEELPANLRIESWVPSQLDVLAHPNVKVFFTHAGG
NGYHEGLYFGKPLVVRPLWVDCDDQAVRGQDFGVSLTVDRPETVDTDD
VLDKITRVLNESSFTERAEYYAGLLKAAGGRTAAADLLLGLPVLAND.

The flexibility and speed provided by cell-free protein synthesis was leveraged to rapidly screen single amino acid substitutions at all sites. To this end, a library was purchased from Twist Bioscience and transformed into standard E. coli expression strain. Colonies were picked and arrayed into a 384-well plate. Colony PCR was performed (1) to provide small linear expression templates for cell-free protein synthesis (CFPS) expression, and (2) for a stock of each DNA for sequencing. CFPS was performed and mannosylation reactions (in the forward direction using GDP-mannose) were performed following the previously described procedure. Reactions were quenched after 12 h with equal volumes of methanol and analyzed by HPLC. Crude percent conversions were calculated for each reaction and a relative activity was calculated based on performance of the wild-type enzyme. Overall, nearly 10,000 reactions were performed and 30 unique single amino acid substitutions were identified to increase activity for the transfer of mannose to AmdeB. Specifically, mutations at residue positions L34, S144, F166, and D407 gave the largest increases in activity, ranging from 2-3.2-fold increases. Recombining mutations at these four positions lead to synergistic gains yielding many triple and quandruple mutants with ˜20-fold increase activity relative to wild-type AmphDI.

### Example 3: AmphDI Triple Mutants S144C-F166N/T-D407F Provide an Expanded Substrate Scope

Having discovered new mutants that catalyzed the transfer of mannose with similar efficiency to that of the wild type enzyme and its natural substrate mycosamine, it was hypothesized that the remodeled activity site may be more permissive. Accordingly, an expanded substrate scope was tested with the top 89 mutants.

### Synthesis of GDP-Sugars

Gram scale synthesis of a small suite of GDP-sugars were biosynthesized following the general protocol outlined in Green Chem., 2021, 23, 2628-2633. GDP-2-fluoromannose, GDP-3-fluoromannose, GDP-4-fluoromannose, GDP-2-chloromannose, GDP-2-deoxymannose, GDP-6-deoxymannose, and GDP-glucose were all prepared in gram scale. Briefly, reaction mixtures containing 200 mM sugar, 600 mM GTP, and 200 MgCl₂ was adjusted to pH 7.5 with 1 M NaOH, followed by a preincubation at 37° C. for 15 minutes before the addition of 2 mg/L BiNahK, 2 mg/L PfManC, and 1 mg/L PmpPA to a final volume of ˜20 mL. The reaction was incubated at 37° C. for 16-28 h. The pH of the reaction was monitored for the first 2-4 h and adjusted using 1 M NaOH to maintain pH 7.5. TLC was used to monitor the formation of GDP-sugar and the consumption of GTP and free sugar. The reactions were quenched by the addition of equal volume of cold ethanol until starting sugar was consumed completely. The purification was the same for all sugars. Appropriate amounts 1 M barium chloride solution was added dropwise to the reaction supernatant (on ice) to remove unreacted nucleotides and byproduct. Insoluble precipitate was removed by centrifugation at 13,000 rpm for 5 min. The above ion precipitation was repeated until no precipitation formed. 200 mL pretreated Amberlite® IRC120 H cation exchange resin (exchange capacity 2 mmol/mL) were added to the supernatant and mixed thoroughly to affinity positively charged impurities, such as Ba²⁺ and Mg²⁺for 30 min at 4° C. A routine filtration process was performed to remove cation exchange resin and the supernatant was lyophilized to generate pure GDP-sugar. GDP-sugar identity and relative purity was assessed by LCMS and used without any further purification steps.

Reaction of GDP-Sugars and AmdeB with the Top 89 Mutants from the GDP-Mannose Campaign to Form Analogues of AmB.

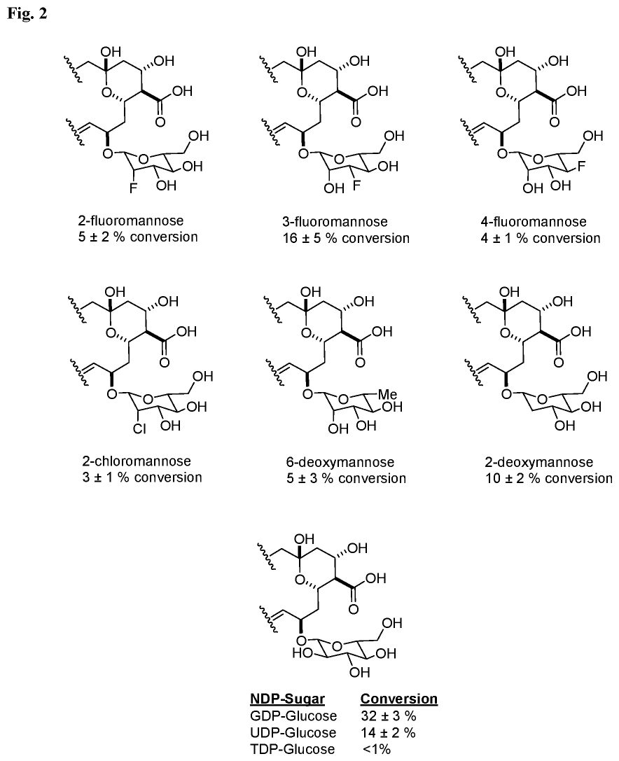

The top 89 mutants (including single, double, triple, and quadruple mutants) were tested with each sugar. Many of the higher order mutants exhibited trace activity with almost all other sugars tested. Two triple mutants, S144C-F166N/T-D407F (SEQ ID NOs: 97 and 98) displayed the most relaxed substrate scope and also displayed activity for both GDP-2-deoxymannose and GDP-glucose. The best conversion for each sugar is summarized in FIG. 2.

To determine whether the mutants were tolerant of other NDPs, UDP-glucose and TDP-glucose were synthesized and tested as substrates. Gratifyingly, the desired glycosylation product was observed with each of the NDPs (FIG. 2)

Reverse reactions were performed on C2′epiAmB with the mutants, but no product was observed.

These results validated the substrate walking approach by successfully overcoming the strict substrate scope of AmphiDI and led to observed activity for intermediate sugars further down our hypothetical path. Additionally, activity was observed for both GDP-2-deoxymannose and GDP-glucose simultaneously. Therefore, in a single round of evolution and with only 3 mutations an intermediate sugar in the hypothetical evolutionary pathway was able to be skipped entirely. Ultimately, two forward campaigns were carried forward, one for 2-deoxymannose and another for glucose, because it's unknown which sugar could lead to the short evolutionary path towards the target sugar.

### Example 4: a Revised Cell-Free Protein Engineering Workflow, GDP-2-Deoxymannose & GDP-Glucose Campaigns

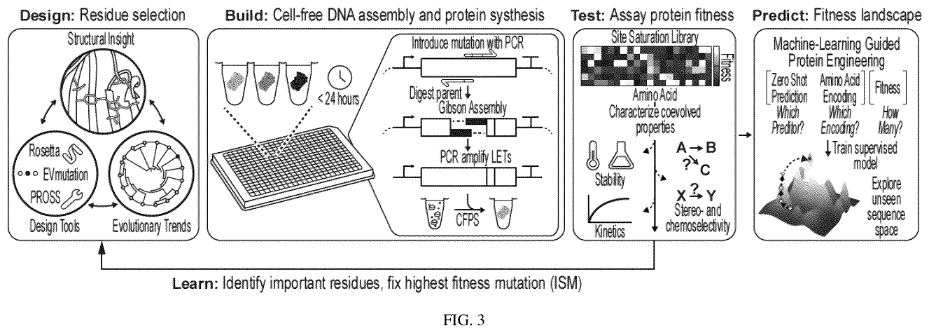

Despite the success of the mannose campaign, analyzing the sequences of only hits and neglecting most mutants that had less than desirable activity data was discouraging. Therefore, in parallel to performing the mannose campaign, a completely cell-free DNA-assembly and protein synthesis enzyme engineering platform was designed to build and test site-saturated, sequence-defined libraries (FIG. 3). After amino acid residue selection based on structural insights, evolutionary trends, and design tools (e.g., ROSETTA, EVmutation, PROSS), the workflow included five steps for high-throughput, cell-free DNA template assembly and expression: (i) a DNA primer containing a mismatch introduces a desired mutation through PCR, (ii) the parent plasmid is digested, (iii) an intramolecular Gibson assembly forms a mutated plasmid, (iv) a second PCR amplifies linear DNA expression templates (LETs), and (v) the mutated protein is expressed through CFPS. In this way, hundreds to thousands of sequence-defined protein mutants can be built and their function can be tested in individual reactions within 24 hours.

This workflow was applied to engineer S144C-F166N/T-D407F (SEQ ID NOs: 97 and 98) for GDP-2-deoxymannose activity. The workflow was implemented in two sequential parts: (1) a hot spot screen (HSS) in which site-saturated mutagenesis was performed on a wide sequence space to identify residue positions that, when mutated, positively impact fitness. (2) Iterative site saturated mutagenesis (ISM) would follow to accumulate beneficial combinations of mutations focused on impactful residue positions identified from the HSS. The same 96 residue positions originally targeted in the mannose screen were ultimately selected, reasoning that in the context of a new substrate and backbone and the sheer coverage of the putative active site would again lead to numerous hits one could recombine using ISM. HSS of these residues (1,825 total unique sequences) revealed 16 potential hot spots. Interestingly, 3 residue positions were previously observed for mannose (positions 32, 144, and 166) and 13 new sites (31, 33, 89, 111, 115, 170, 171, 310, 311, 338, 340, 361, and 382) gave at least 1.3-fold improvements over S144C-F166N/T-D407F. After fixing the top performing mutant from the HSS (1310F), ISM was performed on 8 of the remaining residues identified in the HSS over 4 rounds. Notably, the workflow reintroduces previously fixed mutations to explore potential epistatic interactions. This was a critical decision because two mutations were ultimately revised through ISM; S144C was mutated again to N and F166N/T was mutated again to M. After four rounds of our ISM workflow, no further beneficial mutations were identified and a quintuple mutant (S115T-S144N-F166M-I310F-D407F) was discovered with dramatically increased activity for GDP-2-deoxymannose.

In parallel, a campaign for GDP-glucose activity was performed in the same manner described above. From the same HSS of 1,825 mutants, 16 hot spots were identified. Seven of which were shared with 2-deoxymannose (89, 115, 144, 166, 170, 171, 310), and nine unique positions (85, 112, 241, 242, 285, 307, 400, 401, 407). After four rounds of ISM, a sextuple mutant (V89K-S115T-S144C-N166M-310L-D407F), corresponding to SEQ ID NO. 88, was discovered with dramatically increased activity for GDP-glucose. Similar to the 2-deoxymannose campaign, residue position 166 was revised to M and highlights the need for ISM methods to include residue positions previously screen to maximize results. After the sixth mutation was fixed, subsequent ISM steps failed to final any additional beneficial mutations.

### Example 5: Trace C2′Epimycosamine Activity is Observed

To maximize the chance of success to observe C2′epimycosamine activity, a synthetic GDP-C2′epimycosamine standard was prepared in multi milligram quantities. GDP-C2′epimycosamine was synthesized through a complex chemoenzymatic route involving the synthesis of a para-nitro donor, C2′epimycosamine and a final enzymatic transformation to yield the GDP-C2′epimycosamine (see Gantt et al., PNAS 110 (19), 7648-7653 (2013); https://www.pnas.org/doi/10.1073/pnas.1220220110).

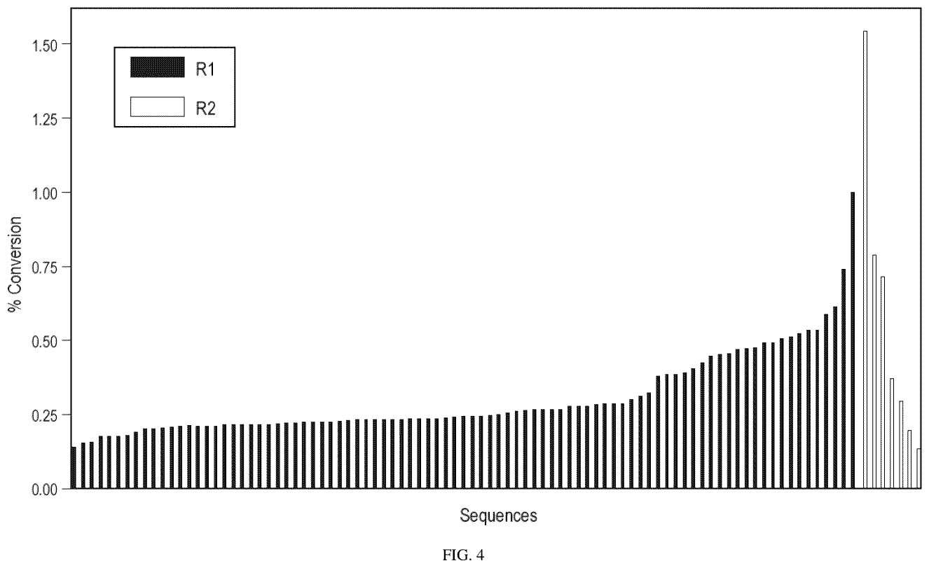

Forward glycosylation reactions were performed with a panel of mutants. Essentially, a final ISM step was performed on the best GDP-glucose backbone using a modified selection of 16 residues. These 16 residue positions were selected based on their proximity to the putative sugar binding site and for their previous influence on activity for any of the 3 intermediate sugars (34, 89, 111, 112, 115, 144, 166, 167, 170, 171, 308, 309, 310, 311, 404, 407). Residues expected to interact with the 3′ and 6′ positions were heavily biased in this selection as these were the only differences between glucose and C2′epimycosamine. The resulting library of 304 unique members plus all previous backbones were assayed for their 2-deoxy, glucose, and C2′epi activity. Surprisingly, several mutants displayed very weak activity for GDP-C2′epimycosamine. The most active mutants, which provided conversions of about 0.13% to about 1.5% (see FIG. 4), comprise the amino acid sequences set forth in SEQ ID NOs: 1-96. The ten most active mutants comprise the amino acid sequences set forth in SEQ ID NOs: 89 (1.54% conversion), 88 (1.0% conversion), 91 (0.79% conversion), 87 (0.74% conversion), 92 (0.72% conversion), 86 (0.61% conversion), 85 (0.59% conversion), 84 (0.53% conversion), 83 (0.53% conversion), and 82 (0.52% conversion). The most active mutants were also tested in the reverse direction using the synthetic C2′epiAmB and yielded small amounts of the expected AmdeB product.

The table below provides the % conversion of AmdeB to C2′epiAmB in the glycosylation reaction described above.

| SEQ ID NO: | % conversion |
|---|---|
| SEQ ID NO: 89 | 1.54320988 |
| SEQ ID NO: 88 | 1 |
| SEQ ID NO: 91 | 0.79012346 |
| SEQ ID NO: 87 | 0.74114441 |
| SEQ ID NO: 92 | 0.71604938 |
| SEQ ID NO: 86 | 0.61307902 |
| SEQ ID NO: 85 | 0.58855586 |
| SEQ ID NO: 84 | 0.53405995 |
| SEQ ID NO: 83 | 0.53405995 |
| SEQ ID NO: 82 | 0.52316076 |
| SEQ ID NO: 81 | 0.51226158 |
| SEQ ID NO: 80 | 0.50681199 |
| SEQ ID NO: 78 | 0.49318801 |
| SEQ ID NO: 79 | 0.49318801 |
| SEQ ID NO: 77 | 0.47683924 |
| SEQ ID NO: 76 | 0.47411444 |
| SEQ ID NO: 75 | 0.47138965 |
| SEQ ID NO: 74 | 0.45776567 |
| SEQ ID NO: 73 | 0.45231608 |
| SEQ ID NO: 72 | 0.44686649 |
| SEQ ID NO: 71 | 0.42506812 |
| SEQ ID NO: 70 | 0.40599455 |
| SEQ ID NO: 69 | 0.39237057 |
| SEQ ID NO: 68 | 0.38692098 |
| SEQ ID NO: 67 | 0.38692098 |
| SEQ ID NO: 66 | 0.38147139 |
| SEQ ID NO: 93 | 0.37037037 |
| SEQ ID NO: 65 | 0.32425068 |
| SEQ ID NO: 64 | 0.3133515 |
| SEQ ID NO: 63 | 0.30245232 |
| SEQ ID NO: 94 | 0.2962963 |
| SEQ ID NO: 62 | 0.28610354 |
| SEQ ID NO: 61 | 0.28610354 |
| SEQ ID NO: 60 | 0.28610354 |
| SEQ ID NO: 59 | 0.28337875 |
| SEQ ID NO: 58 | 0.27792916 |
| SEQ ID NO: 57 | 0.27792916 |
| SEQ ID NO: 56 | 0.27792916 |
| SEQ ID NO: 55 | 0.26702997 |
| SEQ ID NO: 54 | 0.26702997 |
| SEQ ID NO: 53 | 0.26702997 |
| SEQ ID NO: 52 | 0.26702997 |
| SEQ ID NO: 51 | 0.26430518 |
| SEQ ID NO: 50 | 0.26158038 |
| SEQ ID NO: 49 | 0.25613079 |
| SEQ ID NO: 48 | 0.2506812 |
| SEQ ID NO: 47 | 0.2479564 |
| SEQ ID NO: 45 | 0.24523161 |
| SEQ ID NO: 44 | 0.24523161 |
| SEQ ID NO: 46 | 0.24523161 |
| SEQ ID NO: 43 | 0.24250681 |
| SEQ ID NO: 42 | 0.23978202 |
| SEQ ID NO: 41 | 0.23705722 |
| SEQ ID NO: 40 | 0.23705722 |
| SEQ ID NO: 39 | 0.23705722 |
| SEQ ID NO: 38 | 0.23705722 |
| SEQ ID NO: 35 | 0.23433243 |
| SEQ ID NO: 32 | 0.23433243 |
| SEQ ID NO: 34 | 0.23433243 |
| SEQ ID NO: 33 | 0.23433243 |
| SEQ ID NO: 36 | 0.23433243 |
| SEQ ID NO: 37 | 0.23433243 |
| SEQ ID NO: 31 | 0.23160763 |
| SEQ ID NO: 30 | 0.22888283 |
| SEQ ID NO: 29 | 0.22615804 |
| SEQ ID NO: 28 | 0.22615804 |
| SEQ ID NO: 27 | 0.22615804 |
| SEQ ID NO: 26 | 0.22615804 |
| SEQ ID NO: 25 | 0.22343324 |
| SEQ ID NO: 24 | 0.22343324 |
| SEQ ID NO: 23 | 0.22070845 |
| SEQ ID NO: 21 | 0.21798365 |
| SEQ ID NO: 20 | 0.21798365 |
| SEQ ID NO: 22 | 0.21798365 |
| SEQ ID NO: 18 | 0.21525886 |
| SEQ ID NO: 17 | 0.21525886 |
| SEQ ID NO: 19 | 0.21525886 |
| SEQ ID NO: 16 | 0.21253406 |
| SEQ ID NO: 15 | 0.21253406 |
| SEQ ID NO: 14 | 0.21253406 |
| SEQ ID NO: 13 | 0.21253406 |
| SEQ ID NO: 12 | 0.21117166 |
| SEQ ID NO: 11 | 0.20708447 |
| SEQ ID NO: 10 | 0.20435967 |
| SEQ ID NO: 8 | 0.20163488 |
| SEQ ID NO: 9 | 0.20163488 |
| SEQ ID NO: 95 | 0.19753086 |
| SEQ ID NO: 7 | 0.19073569 |
| SEQ ID NO: 6 | 0.17983651 |
| SEQ ID NO: 5 | 0.17711172 |
| SEQ ID NO: 4 | 0.17711172 |
| SEQ ID NO: 3 | 0.17711172 |
| SEQ ID NO: 2 | 0.15803815 |
| SEQ ID NO: 1 | 0.15531335 |
| SEQ ID NO: 90 | 0.14168937 |
| SEQ ID NO: 96 | 0.13580247 |
| SEQ ID NO: 97 | 0 |
| SEQ ID NO: 98 | 0 |

### INCORPORATION BY REFERENCE

All patents and published patent applications mentioned in the description above are incorporated by reference herein in their entirety.

### EQUIVALENTS

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

We claim:

1. A polypeptide, or a salt thereof, comprising an amino acid sequence having at least 95% sequence identity to the amino acid sequence of any one of SEQ ID NOS: 1-98:
(SEQ ID NO: 1)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWKDCF
DQAVRGQDFGVSLTVDRPETVDTDDVLDKITRVLNESSFTERAEYYAGLLKAAGGRTAAADLLLG
LPVLAND;
(SEQ ID NO: 2)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLCRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 3)
GAHRRPILFVSYAESGLLNPLLVLAEELSRRGVEDLWFATDEKARDQIESASADSELQFASLGDT
VSQMSAVTWDDETYAEVTQRSRFKAHRAVHRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 4)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCK
DQAVRGQDFGVSLTVDRPETVDTDDVLDKITRVLNESSFTERAEYYAGLLKAAGGRTAAADLLLG
LPVLAND;
(SEQ ID NO: 5)
GAHRRPILFVSYAESGLLNPLLVLAEELSRRGVEDLWFATDEKARDQIESASADSELQFASLGDT
VSQMSAVTWDDETYAEVTQRSRFKAHRAVKRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 6)
GAHRRPILFVSYAESGLLNPLLVLAEELSRRGVEDLWFATDEKARDQIESASADSELQFASLGDT
VSQMSAVTWDDETYAEVTQRSRFKAHRADIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 7)
GAHRRPILFVSYAESGLLNPLLVLAEELSRRGVEDLWFATDEKARDQIESASADSELQFASLGDT
VSQMSAVTWDDETYAEVTQRSRFKAHRAVCRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 8)
GAHRRPILFVSYAESGLLNPLLVLAEELSRRGVEDLWFATDEKARDQIESASADSELQFASLGDT
VSQMSAVTWDDETYAEVTQRSRFKAHRARIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 9)
GAHRRPILFVSYAESGLLNPLLVLAEELSRRGVEDLWFATDEKARDQIESASADSELQFASLGDT
VSQMSAVTWDDETYAEVTQRSRFKAHRAVPRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 10)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLNRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 11)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWGDCF
DQAVRGQDFGVSLTVDRPETVDTDDVLDKITRVLNESSFTERAEYYAGLLKAAGGRTAAADLLLG
LPVLAND;
(SEQ ID NO: 12)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSRVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 13)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCR
DQAVRGQDFGVSLTVDRPETVDTDDVLDKITRVLNESSFTERAEYYAGLLKAAGGRTAAADLLLG
LPVLAND;
(SEQ ID NO: 14)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCQ
DQAVRGQDFGVSLTVDRPETVDTDDVLDKITRVLNESSFTERAEYYAGLLKAAGGRTAAADLLLG
LPVLAND;
(SEQ ID NO: 15)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCT
DQAVRGQDFGVSLTVDRPETVDTDDVLDKITRVLNESSFTERAEYYAGLLKAAGGRTAAADLLLG
LPVLAND;
(SEQ ID NO: 16)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWRDCF
DQAVRGQDFGVSLTVDRPETVDTDDVLDKITRVLNESSFTERAEYYAGLLKAAGGRTAAADLLLG
LPVLAND;
(SEQ ID NO: 17)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLFRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 18)
GAHRRPILFVSYAESGLLNPLLVLAEELSRRGVEDLWFATDEKARDQIESASADSELQFASLGDT
VSQMSAVTWDDETYAEVTQRSRFKAHRAVSRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 19)
GAHRRPILFVSYAESGVLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 20)
GAHRRPILFVSYAESGLLNPLLVLAEELSRRGVEDLWFATDEKARDQIESASADSELQFASLGDT
VSQMSAVTWDDETYAEVTQRSRFKAHRAVTRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 21)
GAHRRPILFVSYAESGLLNPLLVLAEELSRRGVEDLWFATDEKARDQIESASADSELQFASLGDT
VSQMSAVTWDDETYAEVTQRSRFKAHRAVWRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 22)
GAHRRPILFVSYAESGLLNPLLVLAEELSRRGVEDLWFATDEKARDQIESASADSELQFASLGDT
VSQMSADTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 23)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIEGMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 24)
GAHRRPILFVSYAESGLLNPLLVLAEELSRRGVEDLWFATDEKARDQIESASADSELQFASLGDT
VSQMSAVTWDDETYAEVTQRSRFKAHRAVMRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 25)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLDRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 26)
GAHRRPILFVSYAESGLLNPLLVLAEELSRRGVEDLWFATDEKARDQIESASADSELQFASLGDT
VSQMSAVTWDDETYAEVTQRSRFKAHRAAIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 27)
GAHRRPILFVSYAESGLLNPLLVLAEELSRRGVEDLWFATDEKARDQIESASADSELQFASLGDT
VSQMSAWTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 28)
GAHRRPILFVSYAESGLLNPLLVLAEELSRRGVEDLWFATDEKARDQIESASADSELQFASLGDT
VSQMSAVTWDDETYAEVTQRSRFKAHRAVYRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 29)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWIDCF
DQAVRGQDFGVSLTVDRPETVDTDDVLDKITRVLNESSFTERAEYYAGLLKAAGGRTAAADLLLG
LPVLAND;
(SEQ ID NO: 30)
GAHRRPILFVSYAESGLLNPLLVLAEELSRRGVEDLWFATDEKARDQIESASADSELQFASLGDT
VSQMSAVTWDDETYAEVTQRSRFKAHRATIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 31)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLQRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 32)
GAHRRPILFVSYAESGLLNPLLVLAEELSRRGVEDLWFATDEKARDQIESASADSELQFASLGDT
VSQMSAVTWDDETYAEVTQRSRFKAHRAVGRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 33)
GAHRRPILFVSYAESGLLNPLLVLAEELSRRGVEDLWFATDEKARDQIESASADSELQFASLGDT
VSQMSAVTWDDETYAEVTQRSRFKAHRACIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 34)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPILPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 35)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCH
DQAVRGQDFGVSLTVDRPETVDTDDVLDKITRVLNESSFTERAEYYAGLLKAAGGRTAAADLLLG
LPVLAND;
(SEQ ID NO.: 36)
GAHRRPILFVSYAESGLLNPLLVLAEELSRRGVEDLWFATDEKARDQIESASADSELQFASLGDT
VSQMSAVTWDDETYAEVTQRSRFKAHRAVDRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 37)
GAHRRPILFVSYAESGLLNPLLVLAEELSRRGVEDLWFATDEKARDQIESASADSELQFASLGDT
VSQMSAVTWDDETYAEVTQRSRFKAHRAVIRHAFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 38)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCC
DQAVRGQDFGVSLTVDRPETVDTDDVLDKITRVLNESSFTERAEYYAGLLKAAGGRTAAADLLLG
LPVLAND;
(SEQ ID NO: 39)
GAHRRPILFVSYAESGLLNPLLVLAEELSRRGVEDLWFATDEKARDQIESASADSELQFASLGDT
VSQMSAVTWDDETYAEVTQRSRFKAHRAVQRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 40)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCW
DQAVRGQDFGVSLTVDRPETVDTDDVLDKITRVLNESSFTERAEYYAGLLKAAGGRTAAADLLLG
LPVLAND;
(SEQ ID NO: 41)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWEDCF
DQAVRGQDFGVSLTVDRPETVDTDDVLDKITRVLNESSFTERAEYYAGLLKAAGGRTAAADLLLG
LPVLAND;
(SEQ ID NO: 42)
GAHRRPILFVSYAESGLLNPLLVLAEELSRRGVEDLWFATDEKARDQIESASADSELQFASLGDT
VSQMSAVTWDDETYAEVTQRSRFKAHRAVFRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 43)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCI
DQAVRGQDFGVSLTVDRPETVDTDDVLDKITRVLNESSFTERAEYYAGLLKAAGGRTAAADLLLG
LPVLAND;
(SEQ ID NO: 44)
GAHRRPILFVSYAESGLLNPLLVLAEELSRRGVEDLWFATDEKARDQIESASADSELQFASLGDT
VSQMSAVTWDDETYAEVTQRSRFKAHRAVRRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 45)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCM
DQAVRGQDFGVSLTVDRPETVDTDDVLDKITRVLNESSFTERAEYYAGLLKAAGGRTAAADLLLG
LPVLAND;
(SEQ ID NO: 46)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCD
DQAVRGQDFGVSLTVDRPETVDTDDVLDKITRVLNESSFTERAEYYAGLLKAAGGRTAAADLLLG
LPVLAND;
(SEQ ID NO: 47)
GAHRRPILFVSYAESGLLNPLLVLAEELSRRGVEDLWFATDEKARDQIESASADSELQFASLGDT
VSQMSAVTWDDETYAEVTQRSRFKAHRAVNRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 48)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWLDCF
DQAVRGQDFGVSLTVDRPETVDTDDVLDKITRVLNESSFTERAEYYAGLLKAAGGRTAAADLLLG
LPVLAND;
(SEQ ID NO: 49)
GAHRRPILFVSYAESGLLNPLLVLAEELSRRGVEDLWFATDEKARDQIESASADSELQFASLGDT
VSQMSAVTWDDETYAEVTQRSRFKAHRAVVRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 50)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSAVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 51)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLIRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 52)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCL
DQAVRGQDFGVSLTVDRPETVDTDDVLDKITRVLNESSFTERAEYYAGLLKAAGGRTAAADLLLG
LPVLAND;
(SEQ ID NO: 53)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPHLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 54)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWMDCF
DQAVRGQDFGVSLTVDRPETVDTDDVLDKITRVLNESSFTERAEYYAGLLKAAGGRTAAADLLLG
LPVLAND;
(SEQ ID NO: 55)
GAHRRPILFVSYAESGLLNPLLVLAEELSRRGVEDLWFATDEKARDQIESASADSELQFASLGDT
VSQMSAVTWDDETYAEVTQRSRFKAHRAIIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 56)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGDLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 57)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIERMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 58)
GAHRRPILFVSYAESGLLNPLLVLAEELSRRGVEDLWFATDEKARDQIESASADSELQFASLGDT
VSQMSAVTWDDETYAEVTQRSRFKAHRAVERHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 59)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIESMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 60)
GAHRRPILFVSYAESGLLNPLLVLAEELSRRGVEDLWFATDEKARDQIESASADSELQFASLGDT
VSQMSAVTWDDETYAEVTQRSRFKAHRAVARHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 61)
GAHRRPILFVSYAESGLLNPLLVLAEELSRRGVEDLWFATDEKARDQIESASADSELQFASLGDT
VSQMSATTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 62)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWSDCF
DQAVRGQDFGVSLTVDRPETVDTDDVLDKITRVLNESSFTERAEYYAGLLKAAGGRTAAADLLLG
LPVLAND;
(SEQ ID NO: 63)
GAHRRPILFVSYAESGLLNPLLVLAEELSRRGVEDLWFATDEKARDQIESASADSELQFASLGDT
VSQMSAETWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 64)
GAHRRPILFVSYAESGLLNPLLVLAEELSRRGVEDLWFATDEKARDQIESASADSELQFASLGDT
VSQMSAVTWDDETYAEVTQRSRFKAHRAVLRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 65)
GAHRRPILFVSYAESGLLNPLLVLAEELSRRGVEDLWFATDEKARDQIESASADSELQFASLGDT
VSQMSAVTWDDETYAEVTQRSRFKAHRAMIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 66)
GAHRRPILFVSYAESGLLNPLLVLAEELSRRGVEDLWFATDEKARDQIESASADSELQFASLGDT
VSQMSAVTWDDETYAEVTQRSRFKAHRALIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 67)
GAHRRPILFVSYAESGLLNPLLVLAEELSRRGVEDLWFATDEKARDQIESASADSELQFASLGDT
VSQMSAHTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 68)
GAHRRPILFVSYAESGLLNPLLVLAEELSRRGVEDLWFATDEKARDQIESASADSELQFASLGDT
VSQMSALTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 69)
GAHRRPILFVSYAESGLLNPLLVLAEELSRRGVEDLWFATDEKARDQIESASADSELQFASLGDT
VSQMSAYTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 70)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIEAMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 71)
GAHRRPILFVSYAESGLLNPLLVLAEELSRRGVEDLWFATDEKARDQIESASADSELQFASLGDT
VSQMSANTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 72)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIEHMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 73)
GAHRRPILFVSYAESGLLNPLLVLAEELSRRGVEDLWFATDEKARDQIESASADSELQFASLGDT
VSQMSAFTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 74)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPRLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 75)
GAHRRPILFVSYAESGLLNPLLVLAEELSRRGVEDLWFATDEKARDQIESASADSELQFASLGDT
VSQMSAVTWDDETYAEVTQRSRFKAHRAVIRHCFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 76)
GAHRRPILFVSYAESGLLNPLLVLAEELSRRGVEDLWFATDEKARDQIESASADSELQFASLGDT
VSQMSACTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 77)
GAHRRPILFVSYAESGLLNPLLVLAEELSRRGVEDLWFATDEKARDQIESASADSELQFASLGDT
VSQMSAQTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 78)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPLLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 79)
GAHRRPILFVSYAESGLLNPLLVLAEELSRRGVEDLWFATDEKARDQIESASADSELQFASLGDT
VSQMSAITWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 80)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPALPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 81)
GAHRRPILFVSYAESGLLNPLLVLAEELSRRGVEDLWFATDEKARDQIESASADSELQFASLGDT
VSQMSASTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 82)
GAHRRPILFVSYAESGLLNPLLVLAEELSRRGVEDLWFATDEKARDQIESASADSELQFASLGDT
VSQMSAATWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 83)
GAHRRPILFVSYAESGLLNPLLVLAEELSRRGVEDLWFATDEKARDQIESASADSELQFASLGDT
VSQMSAGTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 84)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMNPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 85)
GAHRRPILFVSYAESGLLNPLLVLAEELSRRGVEDLWFATDEKARDQIESASADSELQFASLGDT
VSQMSAMTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 86)
GAHRRPILFVSYAESGLLNPLLVLAEELSRRGVEDLWFATDEKARDQIESASADSELQFASLGDT
VSQMSAVTWDDETYAEVTQRSRFKAHRAVIRHTFAPETRVEKYRALEKAVEEIQPALMVIECMCQ
FGYELAITKGIPFVLGVPMAPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 87)
GAHRRPILFVSYAESGLLNPLLVLAEELSRRGVEDLWFATDEKARDQIESASADSELQFASLGDT
VSQMSARTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 88)
GAHRRPILFVSYAESGLLNPLLVLAEELSRRGVEDLWFATDEKARDQIESASADSELQFASLGDT
VSQMSAKTWDDETYAEVTQRSRFKAHRAVIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 89)
GAHRRPILFVSYAESGLLNPLLVLAEELSRRGVEDLWFATDEKARDQIESASADSELQFASLGDT
VSQMSAMTWDDETYAEVTQRSRFKAHRAVQRHTFAPETRVEKYRALEKAVEEIQPALMVIEHMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCR
DQAVRGQDFGVSLTVDRPETVDTDDVLDKITRVLNESSFTERAEYYAGLLKAAGGRTAAADLLLG
LPVLAND;
(SEQ ID NO: 90)
GAHRRPILFVSYAESGLLNPLLVLAEELSRRGVEDLWFATDEKARDQIESASADSELQFASLGDT
VSQMSAVTWDDETYAEVTQRSRFKAHRASIRHTFAPETRVEKYRALEKAVEEIQPALMVIECMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 91)
GAHRRPILFVSYAESGLLNPLLVLAEELSRRGVEDLWFATDEKARDQIESASADSELQFASLGDT
VSQMSAKTWDDETYAEVTQRSRFKAHRAVARHTFAPETRVEKYRALEKAVEEIQPALMVIECMCQ
FGYELAITKGIPFVLGVPALPSAVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCD
DQAVRGQDFGVSLTVDRPETVDTDDVLDKITRVLNESSFTERAEYYAGLLKAAGGRTAAADLLLG
LPVLAND;
(SEQ ID NO: 92)
GAHRRPILFVSYAESGLLNPLLVLAEELSRRGVEDLWFATDEKARDQIESASADSELQFASLGDT
VSQMSAMTWDDETYAEVTQRSRFKAHRAVIRHTFAPETRVEKYRALEKAVEEIQPALMVIEHMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCM
DQAVRGQDFGVSLTVDRPETVDTDDVLDKITRVLNESSFTERAEYYAGLLKAAGGRTAAADLLLG
LPVLAND;
(SEQ ID NO: 93)
GAHRRPILFVSYAESGLLNPLLVLAEELSRRGVEDLWFATDEKARDQIESASADSELQFASLGDT
VSQMSAMTWDDETYAEVTQRSRFKAHRAVARHTFAPETRVEKYRALEKAVEEIQPALMVIEHMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 94)
GAHRRPILFVSYAESGLLNPLLVLAEELSRRGVEDLWFATDEKARDQIESASADSELQFASLGDT
VSQMSAMTWDDETYAEVTQRSRFKAHRAVIRHTFAPETRVEKYRALEKAVEEIQPALMVIEHMCQ
FGYELAITKGIPFVLGVPMLPSLVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 95)
GAHRRPILFVSYAESGLLNPLLVLAEELSRRGVEDLWFATDEKARDQIESASADSELQFASLGDT
VSQMSAMTWDDETYAEVTQRSRFKAHRAVERHTFAPETRVEKYRALEKAVEEIQPALMVIEHMCQ
FGYELAITKGIPFVLGVPMLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCL
DQAVRGQDFGVSLTVDRPETVDTDDVLDKITRVLNESSFTERAEYYAGLLKAAGGRTAAADLLLG
LPVLAND;
(SEQ ID NO: 96)
GAHRRPILFVSYAESGLLNPLLVLAEELSRRGVEDLWFATDEKARDQIESASADSELQFASLGDT
VSQMSAMTWDDETYAEVTQRSRFKAHRAVIRHTFAPETRVEKYRALEKAVEEIQPALMVIEHMCQ
FGYELAITKGIPFVLGVPLLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTLTRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
(SEQ ID NO: 97)
GAHRRPILFVSYAESGLLNPLLVLAEELSRRGVEDLWFATDEKARDQIESASADSELQFASLGDT
VSQMSAVTWDDETYAEVTQRSRFKAHRAVIRHSFAPETRVEKYRALEKAVEEIQPALMVIECMCQ
FGYELAITKGIPFVLGVPNLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTITRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND;
and
(SEQ ID NO: 98)
GAHRRPILFVSYAESGLLNPLLVLAEELSRRGVEDLWFATDEKARDQIESASADSELQFASLGDT
VSQMSAVTWDDETYAEVTQRSRFKAHRAVIRHSFAPETRVEKYRALEKAVEEIQPALMVIECMCQ
FGYELAITKGIPFVLGVPTLPSNVLTSHVPFAKSYTPSGFPVPHSGLPGKMSLAQRVENELFRVR
TLGMFMTKEIREIVEEDNRVRGELGISPEARQMMARIDHAEQVLCYSVAELDYPFPMHEKVRLVG
TLVPPLPQAPDDEGLSDWLTEQKSVVFMGFGTITRLTREQVASLVEVARRLEGEGHQVLWKLPSE
QQHLLPPAEELPANLRIESWVPSQLDVLAHPNVKVFFTHAGGNGYHEGLYFGKPLVVRPLWVDCF
DQAVRGQDFGVSLTVDRPETVDTDDVLDKITRVLNESSFTERAEYYAGLLKAAGGRTAAADLLLG
LPVLAND.

2. The polypeptide of claim 1, comprising an amino acid sequence having at least 96% sequence identity to the amino acid sequence of any one of SEQ ID NOs.: 1-98.

3. The polypeptide of claim 1, comprising an amino acid sequence having at least 97% sequence identity to the amino acid sequence of any one of SEQ ID NOs.: 1-98.

4. The polypeptide of claim 1, comprising an amino acid sequence having at least 98% sequence identity to the amino acid sequence of any one of SEQ ID NOs.: 1-98.

5. The polypeptide of claim 1, comprising an amino acid sequence having at least 99% sequence identity to the amino acid sequence of any one of SEQ ID NOs.: 1-98.

6. The polypeptide of claim 1, comprising an amino acid sequence having at least 95% sequence identity to the amino acid sequence of any one of SEQ ID NOs.: 73-89, 91, and 92.

7. The polypeptide of claim 1, comprising an amino acid sequence having at least 95% sequence identity to the amino acid sequence of any one of SEQ ID NOs.: 86-89, 91, and 92.

8. The polypeptide of claim 1, comprising an amino acid sequence having at least 95% sequence identity to the amino acid sequence of any one of SEQ ID NOs.: 87-89 and 91.

9. The polypeptide of claim 1, comprising an amino acid sequence having at least 95% sequence identity to the amino acid sequence of SEQ ID NO: 88.

10. The polypeptide of claim 9, comprising an amino acid sequence having at least 96% sequence identity to the amino acid sequence of SEQ ID NO: 88.

11. The polypeptide of claim 9, comprising an amino acid sequence having at least 97% sequence identity to the amino acid sequence of SEQ ID NO: 88.

12. The polypeptide of claim 9, comprising an amino acid sequence having at least 98% sequence identity to the amino acid sequence of SEQ ID NO: 88.

13. The polypeptide of claim 9, comprising an amino acid sequence having at least 99% sequence identity to the amino acid sequence of SEQ ID NO: 88.

14. The polypeptide of claim 1, comprising an amino acid sequence having at least 95% sequence identity to the amino acid sequence of SEQ ID NO: 89.

15. The polypeptide of claim 14, comprising an amino acid sequence having at least 96% sequence identity to the amino acid sequence of SEQ ID NO: 89.

16. The polypeptide of claim 14, comprising an amino acid sequence having at least 97% sequence identity to the amino acid sequence of SEQ ID NO: 89.

17. The polypeptide of claim 14, comprising an amino acid sequence having at least 98% sequence identity to the amino acid sequence of SEQ ID NO: 89.

18. The polypeptide of claim 14, comprising an amino acid sequence having at least 99% sequence identity to the amino acid sequence of SEQ ID NO: 89.

19. A method of glycosylating the C19 hydroxyl group of AmdeB, comprising the step of combining under conditions sufficient to glycosylate the C19 hydroxyl group of AmdeB:
(i) AmdeB, or a salt thereof; 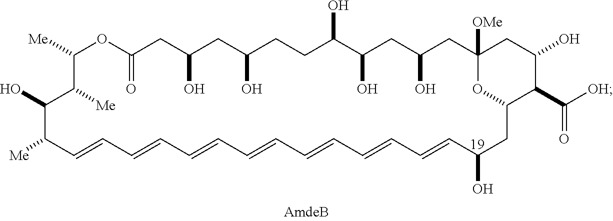
(ii) a saccharide selected from the group consisting of: 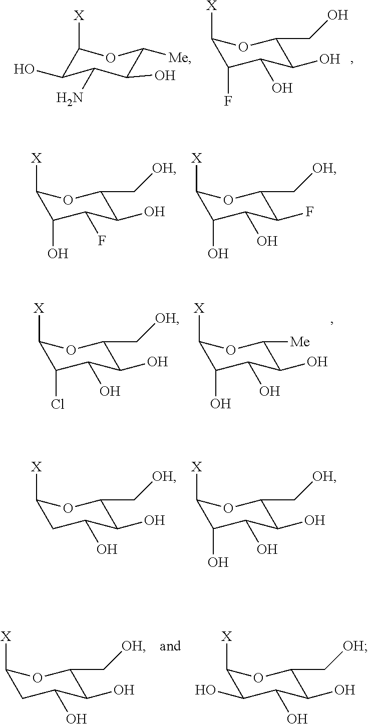
wherein X is an oxygen-linked nucleoside diphosphate; and
(iii) a polypeptide, or a salt thereof, comprising an amino acid sequence having at least 95% sequence identity to the amino acid sequence of any one of SEQ ID NOs.: 1-98.

20. The method of claim 19, wherein X is oxygen-linked guanosine diphosphate, adenosine diphosphate, cytosine diphosphate, uridine diphosphate, or thymidine diphosphate.

21. The method of claim 19 or 20, wherein X is oxygen-linked guanosine diphosphate.

22. The method of any one of claims 19-21, wherein the saccharide is

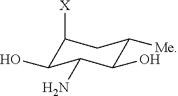

23. The method of any one of claims 19-22, wherein the molar ratio of the saccharide to the polypeptide is from about 10,000:1 to about 100:1.

24. The method of any one of claims 19-23, wherein the molar ratio of AmdeB to the polypeptide is from about 10:1 to about 20:1.

25. A compound, or a pharmaceutically acceptable salt thereof, selected from the group consisting of:

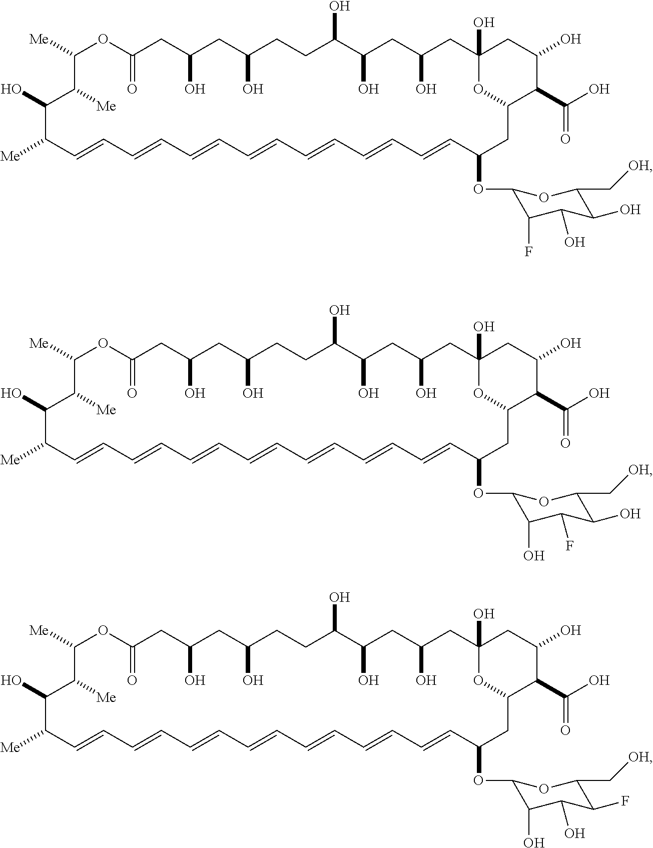

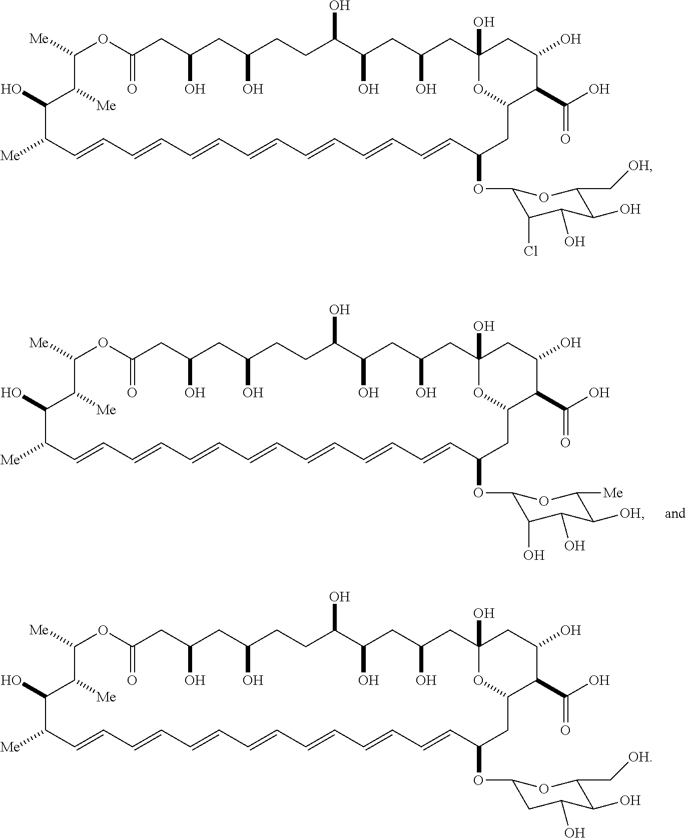

26. The compound of claim 25, selected from the group consisting of:

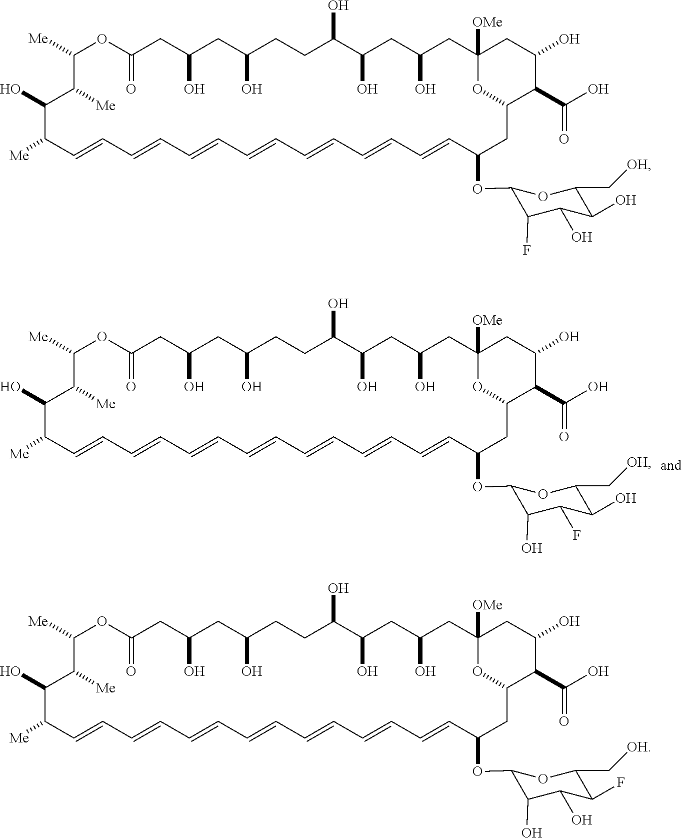

27. The compound of claim 25, selected from the group consisting of:

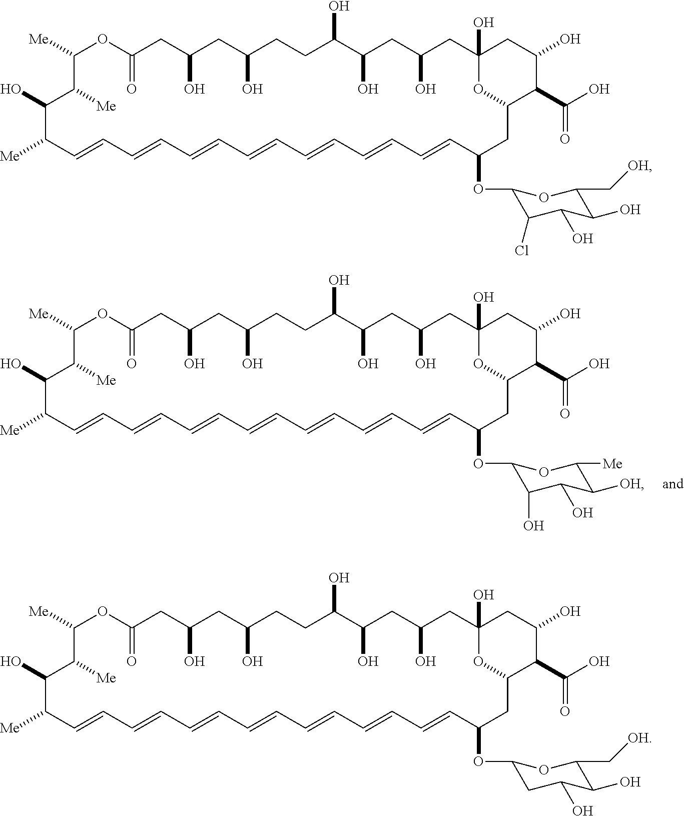

28. A pharmaceutical composition, comprising a compound of any one of claims 25-27; and a pharmaceutically acceptable carrier.
